(19) United States
(12) Patent Application Publication
(10) Pub. No.: US 2005/0079612 A1
(43) Pub. Date: Apr. 14, 2005

(54) Use of obg3 for promoting central nervous system remyelination

(76) Inventor: John Lucas, Concord, MA (US)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 10/495,317

(22) PCT Filed: Oct. 29, 2002
(86) PCT No.: PCT/IB02/04924

Related U.S. Application Data
(60) Provisional application No. 60/332,119, filed on Nov. 21, 2001.

(51) Int. Cl. A61K 38/17; C12N 5/08; C12N 15/85

(52) U.S. Cl. 435/455; 435/368; 514/12

(57) ABSTRACT

The present invention relates to the field of central nervous system (CNS) research. Demyelination of neuronal axons within the CNS underlies the pathogenesis of degenerative diseases of the neuromuscular system, such as multiple sclerosis and hereditary leukodystrophies. Therefore, treatments aimed towards accelerating the repair of myelin sheaths offer a potential therapeutic to ameliorate the symptoms of multiple sclerosis and leukodystrophies. A compound, globular OBG3, has been identified that has immunosuppressive properties. This compound should be effective for accelerating the rate of remyelination and treating multiple sclerosis and leukodystrophies.

### RELATED APPLICATION INFORMATION

This application claims priority on U.S. provisional patent application Ser. No. 60/332,119, filed Nov. 21, 2000, entitled “Use of OBG3 for promoting central nervous system remyelination”.

### FIELD OF THE INVENTION

The present invention relates to the field of central nervous system research, in particular the discovery of compounds effective for accelerating the repair of myelin sheaths of demyelinated neurons and useful for treating multiple sclerosis and hereditary leukodystrophies.

### BACKGROUND OF THE INVENTION

The following discussion is intended to facilitate the understanding of the invention, but is not intended nor admitted to be prior art to the invention.

Demyelinating diseases are those in which myelin is the primary target They fall into two main groups: acquired diseases and hereditary metabolic disorders. The most common acquired disease is multiple sclerosis (MS), a chronic, frequently progressive, inflammatory central nervous system (CNS) disease characterized pathologically by primary demyelination, usually without initial axonal injury. The etiology and pathogenesis of MS are unknown. Several immunological features of MS, and its moderate association with certain major histocompatibility complex alleles, have prompted the speculation that MS is an immune-mediated disease.

The hereditary metabolic disorders include eight identified leukodystrophies: metachromatic leukodystrophy, Refsum's disease, adrenoleukodystrophy, Krabbe's disease, phenylketonuria, Canavan disease, Pelizaeus-Merzbacher disease, and Alexander's disease. The first six are storage disorders. The lack or the malfunctioning of an enzyme causes a toxic buildup of chemical substances. The etiology of Pelizaeus-Merzbacher disease and Alexander's disease, on the other hand, remains unknown.

The limited efficacy of current therapies for MS and other demyelinating diseases has stimulated interest in novel therapies to ameliorate these diseases. However, due to the apparently complex etiopathogenesis of these diseases, potentially involving both environmental and autoimmune factors, the need still exists for an effective treatment of these demyelinating disorders.

### SUMMARY OF THE INVENTION

The instant invention is based on the discovery that portions of the full-length OBG3 polypeptide, termed OBG3 polypeptide fragments or gOBG3 polypeptide fragments, have unexpected effects in vitro and in vivo, including utility for remyelination and immunosuppression in humans and other mammals. These unexpected effects of OBG3 or gOBG3 polypeptide fragment administration in mammals also include reduction of elevated free fatty acid levels caused by administration of epinephrine, i.v. injection of “intralipid”, or administration of a high fat test meal, as well as increased fatty acid oxidation in muscle cells, and weight reduction in mammals consuming a high fat/high sucrose diet. These effects are unexpected and surprising given that administration of full-length OBG3 polypeptide typically has no effect or a significantly reduced effect in vivo or in vitro depending on the specific biological activity and the amount administered. To the extent that any effect is observed following administration of full-length OBG3 polypeptide, the levels of full-length OBG3 polypeptide required for an effect render it unfeasible in most instances as a potential treatment for humans at this time. In contrast, the OBG3 and gOBG3 polypeptide fragments of the invention are radically more effective and thus can be provided at levels that are feasible for treatments in humans.

Thus, the invention is drawn to OBG3 and gOBG3 polypeptide fragments, polynucleotides encoding said OBG3 and gOBG3 polypeptide fragments, vectors comprising said OBG3 and gOBG3 polynucleotides, and cells recombinant for said OBG3 and gOBG3 polynucleotides, as well as to pharmaceutical and physiologically acceptable compositions comprising said OBG3 and gOBG3 polypeptide fragments and methods of administering said OBG3 and gOBG3 pharmaceutical and physiologically acceptable compositions in order to accelerate the rate of remyelination in the central nervous system.

In a first aspect, the invention features a purified, isolated, or recombinant OBG3 or gOBG3 polypeptide fragment that that has significantly greater activity than a full-length OBG3 polypeptide, wherein said activity is accelerating the rate of remyelination of demyelinated neurons in a mammal or human. In preferred embodiments, said polypeptide fragment comprises, consists essentially of, or consists of, at least 6 and not more than 238 consecutive amino acids of SEQ ID NO:6 or at least 6 and not more than 241 consecutive amino acids of SEQ ID NO:2 or SEQ ID NO:4. In other preferred embodiments, OBG3 or gOBG3 polypeptide fragments having unexpected activity are selected from amino acids 84-244, 85-244, 86-244, 87-244, 88-244, 89-244, 90-244, 91-244, 92-244, 93-244, 94-244, 95-244, 96-244, 97-244, 98-244, 99-244, 100-244, 101-244, 102-244, or 103-244 of SEQ ID NO:6. In other preferred embodiments, OBG3 or gOBG3 polypeptide fragments having unexpected activity are selected from amino acids 88-247, 89-247, 90-247, 91-247, 92-247, 93-247, 94-247, 95-247, 96-247, 97-247, 98-247, 99-247, 100-247, 101-247, 102-247, 103-247, 104-247, 105-247, or 106-247 of SEQ ID NO:2 or SEQ ID NO:4. In other preferred embodiments, OBG3 or gOBG3 polypeptide fragments are selected from amino acids about 84 to 244, 85 to 244, 101 to 244, 102 to 244, or 103 to 244 of SEQ ID NO:6 and amino acids 88 to 247, 104 to 247, 105 to 247, or 106 to 247 of SEQ ID NO:2 or SEQ ID NO:4. In further preferred embodiments, gOBG3 polypeptide fragments are said selected gOBG3 polypeptide fragments made resistant to dipeptidyl peptidase cleavage by N-terminal modification. In other further preferred embodiments, said polypeptide fragment comprises an amino acid sequence at least 70%, 75%, 80%, 85%, 90%, 91%, 92%, 93%, 94%, 95%, 96%, 97%, 98%, or 99% identical to the corresponding consecutive amino acids of SEQ ID NO:6, SEQ ID NO:2 or SEQ ID NO:4.

In other highly preferred embodiments, said polypeptide fragment comprises, consists essentially of, or consists of, a purified, isolated, or recombinant gOBG3 fragment. Preferably, said gOBG3 polypeptide fragment comprises, consists essentially of, or consists of, at least 6 consecutive amino acids of amino acids 84 to 244 of SEQ ID NO:6 or at least 6 consecutive amino acids of amino acids 88 to 247 of SEQ ID NO:2 or SEQ ID NO:4. In other preferred embodiments, gOBG3 polypeptide fragments having unexpected activity are selected from amino acids 84-244, 85-244, 86-244, 87-244, 88-244, 89-244, 90-244, 91-244, 92-244, 93-244, 94-244, 95-244, 96-244, 97-244, 98-244, 99-244, 100-244, 101-244, 102-244, or 103-244 of SEQ ID NO:6. In other preferred embodiments, gOBG3 polypeptide fragments having unexpected activity are selected from amino acids 88-247, 89-247, 90-247, 91-247, 92-247, 93-247, 94-247, 95-247, 96-247, 97-247, 98-247, 99-247, 100-247, 101-247, 102-247, 103-247, 104-247, 105-247, or 106-247 of SEQ ID NO:2 or SEQ ID NO:4. In other preferred embodiments, gOBG3 polypeptide fragments are selected from amino acids 84 to 244, 85 to 244, 101 to 244, 102 to 244, or 103 to 244 of SEQ ID NO:6 and amino acids 88 to 247, 104 to 247, 105 to 247, or 106 to 247 of SEQ ID NO:2 or SEQ ID NO:4. In further preferred embodiments, gOBG3 polypeptide fragments are said selected gOBG3 polypeptide fragments made resistant to dipeptidyl peptidase cleavage by N-terminal modification. Alternatively, said gOBG3 fragment comprises, consists essentially of, or consists of, an amino acid sequence at least 90%, 91%, 92%, 93%, 94%, 95%, 96%, 97%, 98%, or 99% identical to the corresponding amino acids 84 to 244 of SEQ ID NO:6 or at least 75% identical to amino acids 88 to 247 of SEQ ID NO:2 or SEQ ID NO:4. In a further preferred embodiment, the OBG3 polypeptide fragment is identical to an APM1 proteolytic cleavage product from human plasma. Preferably, the proteolytic cleavage product comprises the C1q globular head or a portion thereof. More preferably, the proteolytic cleavage product is identical to a proteolytic cleavage product isolated from human plasma by immunoprecipitation using antibodies specific for the C1q globular head. More preferably the proteolytic cleavage product cannot be immunoprecipitated from human plasma using an antibody directed against the human non-homologous region (HDQETITQGPGVLLPLPKGA) of APM1. Still more preferably, the APM1 proteolytic cleavage product has an apparent molecular weight of 27 kDa using SDS-PAGE.

In another aspect, the present invention is also directed towards poylpeptide fragments of OBG3 having biological activity. Preferred polypeptide fragment is amino acids 166-193 or further fragments thereof. Particularly preferred polypeptide fragment is amino acids 166-176. Other particularly preferred fragment is 167-176.

In a further preferred embodiment, the OBG3 or gOBG3 polypeptide fragment is able to increase the rate of cellular division of oligodendrocyte progenitor cells in a mammal or human.

Further preferred OBG3 or gOBG3 polypeptide fragments are those that increase the rate of migration of oligodendrocyte progenitor cells from the germinal centers to demyelinated axons in the CNS of a mammal or human.

Further preferred OBG3 or gOBG3 polypeptide fragments are those that induce oligodendrocyte progenitor cells to differentiate into mature dendrocytic cells at sites of demyelination in axons of in the CNS of a mammal or a human.

Further preferred OBG3 or gOBG3 polypeptide fragments are those that significantly reduce or eliminate the symptoms of multiple sclerosis in a mammal or human. Further preferred OBG3 or gOBG3 polypeptide fragments are those that significantly reduce or eliminate the symptoms of hereditary leukodystrophies, which include metachromatic leukodystrophy, Refsum's disease, adrenoleukodystrophy, Krabbe's disease, phenylketonuria, Canavan disease, Pelizaeus-Merzbacher disease, and Alexander's disease.

Further preferred OBG3 or gOBG3 polypeptide fragments are those that form multimers (e.g., heteromultimers or homomultimers) in vitro and/or in vivo. Preferred multimers are homodimers or homotrimers. Other preferred multimers are homomultimers comprising at least 4, 6, 8, 9, 10, or 12 OBG3 or gOBG3 polypeptide fragment subunits. Other preferred mulimers are hetero multimers comprising a OBG3 or gOBG3 polypeptide fragment of the invention.

Further preferred embodiments include heterologous polypeptides comprising an OBG3 or gOBG3 polypeptide fragment of the invention.

In a further embodiment, the invention features a pharmaceutical or physiologically acceptable composition comprising, consisting essentially of, or consisting of, said OBG3 or gOBG3 polypeptide fragment described in the first aspect and, alternatively, a pharmaceutical or physiologically acceptable diluent.

In a further embodiment, the invention features a method for the production of a pharmaceutical or physiologically acceptable composition consisting essentially of, or consisting of, said OBG3 or gOBG3 polypeptide fragment described in the first aspect and, alternatively, a pharmaceutical or physiologically acceptable diluent.

Full-length OBG3 (ACRP30, AdipoQ, APM1) polypeptides and polynucleotides encoding the same may be specifically substituted for an OBG3 or gOBG3 polypeptide fragment or polynucleotide encoding the same in any embodiment of the present invention.

### PREFERRED EMBODIMENTS OF THE INVENTION

I. OBG3 Polypeptide Fragments of the Invention

The invention is drawn, inter alia, to isolated, purified or recombinant OBG3 polypeptide fragments. OBG3 polypeptide fragments of the invention are useful for accelerating the regeneration of myelin sheaths in vivo. OBG3 polypeptide fragments are also useful inter alia in screening assays for agonists or antagonists of OBG3 fragment activity; in screening assays for antagonists of dipeptidyl peptidase cleavage of OBG3 fragments, preferably cleavage of the N-terminal EP dipeptide of OBG3 polypeptide fragment 103-244 of SEQ ID NO:6, cleavage of the N-terminal VP dipeptide of OBG3 fragment 85-244 of SEQ ID NO:6, or cleavage of the N-terminal EP dipeptide of OBG3 polypeptide fragment 106-247 of SEQ ID NO:2 or SEQ ID NO:4; for raising OBG3 fragment-specific antibodies; and in diagnostic assays.

The full-length OBG3 polypeptide is comprised of at least four distinct regions including:
1. an N-terminal putative signal sequence about from amino acids 1-17 of SEQ ID NO:6, SEQ ID NO:2, or SEQ ID NO:4;
2. a unique region about from amino acids 18-41 of SEQ ID NO:6 or 18-44 of SEQ ID NO:2, or SEQ ID NO:4;
3. a collagen-like region about from amino acids 42-107 of SEQ ID NO:6 or 45-110 of SEQ ID NO:2 or SEQ ID NO:4; and
4. a globular region about from amino acids 108-244 of SEQ ID NO:6 or 111-247 of SEQ ID NO:2 or SEQ ID NO:4.

The term “collagen residues” is used in the manner standard in the art to mean the amino acid triplet glycine, X, Y, where X and Y can be any amino acid.

The OBG3 polypeptide fragments of the present invention are preferably provided in an isolated form, and may be partially or substantially purified. A recombinantly produced version of an OBG3 polypeptide fragment can be substantially purified by the one-step method described by Smith et al. ((1988) Gene 67(1):31-40) or by the methods described herein or known in the art (see, e.g., Examples 1-3). Fragments of the invention also can be purified from natural or recombinant sources using antibodies directed against the polypeptide fragments of the invention by methods known in the art of protein purification.

Preparations of OBG3 polypeptide fragments of the invention involving a partial purification of or selection for the OBG3 polypeptide fragments are also specifically contemplated. These crude preparations are envisioned to be the result of the concentration of cells expressing OBG3 polypeptide fragments with perhaps a few additional purification steps, but prior to complete purification of the fragment. The cells expressing OBG3 polypeptide fragments are present in a pellet, they are lysed, or the crude polypeptide is lyophilized, for example.

OBG3 or gOBG3 polypeptide fragments can be any integer in length from at least 6 consecutive amino acids to 1 amino acid less than a full-length OBG3 polypeptide. Thus, for human OBG3 (SEQ ID NO: 6), an OBG3 or gOBG3 polypeptide fragment can be any integer of consecutive amino acids from 6 to 243; for mouse OBG3 (SEQ ID NO:2 or SEQ ID NO:4) an OBG3 or gOBG3 fragment can be any integer of consecutive amino acids from 6 to 246, for example. The term “integer” is used herein in its mathematical sense and thus representative integers include: 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 234, 235, 236, 237, 238, 239, 240, 241, 242, 243, 244, 245, and 246.

Each OBG3 fragment as described above can be further specified in terms of its N-terminal and C-terminal positions. For example, every combination of a N-terminal and C-terminal position that fragments of from 6 contiguous amino acids to 1 amino acids less than the full-length OBG3 polypeptide could occupy, on any given intact and contiguous full-length OBG3 polypeptide sequence are included in the present invention. Thus, a 6 consecutive amino acid fragment could occupy positions selected from the group consisting of 1-6, 2-7, 3-8, 4-9, 5-10, 6-11, 7-12, 8-13, 9-14, 10-15, 11-16, 12-17, 13-18, 14-19, 15-20, 16-21, 17-22, 18-23, 19-24, 20-25, 21-26, 22-27, 23-28, 24-29, 25-30, 26-31, 27-32, 28-33, 29-34, 30-35, 31-36, 32-37, 33-38, 34-39, 35-40, 36-41, 37-42, 38-43, 39-44, 40-45, 41-46, 42-47, 43-48, 44-49, 45-50, 46-51, 47-52, 48-53, 49-54, 50-55, 51-56, 52-57, 53-58, 54-59, 55-60, 56-61, 57-62, 58-63, 59-64, 60-65, 61-66, 62-67, 63-68, 64-69, 65-70, 66-71, 67-72, 68-73, 69-74, 70-75, 71-76, 72-77, 73-78, 74-79, 75-80, 76-81, 77-82, 78-83, 79-84, 80-85, 81-86, 82-87, 83-88, 84-89, 85-90, 86-91, 87-92, 88-93, 89-94, 90-95, 91-96, 92-97, 93-98, 94-99, 95-100, 96-101, 97-102, 98-103, 99-104, 100-105, 101-106, 102-107, 103-108, 104-109, 105-110, 106-111, 107-112, 108-113, 109-114, 110-115, 111-116, 112-117, 113-118, 114-119, 115-120, 116-121, 117-122, 118-123, 119-124, 120-125, 121-126, 122-127, 123-128, 124-129, 125-130, 126-131, 127-132, 128-133, 129-134, 130-135, 131-136, 132-137, 133-138, 134-139, 135-140, 136-141, 137-142, 138-143, 139-144, 140-145, 141-146, 142-147, 143-148, 144-149, 145-150, 146-151, 147-152, 148-153, 149-154, 150-155, 151-156, 152-157, 153-158, 154-159, 155-160, 156-161, 157-162, 158-163, 159-164, 160-165, 161-166, 162-167, 163-168, 164-169, 165-170, 166-171, 167-172, 168-173, 169-174, 170-175, 171-176, 172-177, 173-178, 174-179, 175-180, 176-181, 177-182, 178-183, 179-184, 180-185, 181-186, 182-187, 183-188, 184-189, 185-190, 186-191, 187-192, 188-193, 189-194, 190-195, 191-196, 192-197, 193-198, 194-199, 195-200, 196-201, 197-202, 198-203, 199-204, 200-205, 201-206, 202-207, 203-208, 204-209, 205-210, 206-211, 207-212, 208-213, 209-214, 210-215, 211-216, 212-217, 213-218, 214-219, 215-220, 216-221, 217-222, 218-223, 219-224, 220-225, 221-226, 222-227, 223-228, 224-229, 225-230, 226-231, 227-232, 228-233, 229-234, 230-235, 231-236, 232-237, 233-238, 234-239, 235-240, 236-241, 237-242, 238-243, and 239-244 of SEQ ID NO:6. A 238 consecutive amino acid fragment could occupy positions selected from the group consisting of 1-238, 2-239, 3-240, 4-241, 5-242, 6-243 and 7-244 of SEQ ID NO:6. Similarly, the positions occupied by all the other fragments of sizes between 6 amino acids and 243 amino acids on SEQ ID NO:6 are included in the present invention and can also be immediately envisaged based on these two examples and therefore, are not individually listed solely for the purpose of not unnecessarily lengthening the specification. Furthermore, the positions occupied by fragments of 6 to 241 consecutive amino acids on SEQ ID NO:2 or SEQ ID NO:4 are included in the present invention and can also be immediately envisaged based on these two examples and therefore are not individually listed solely for the purpose of not unnecessarily lengthening the specification. In addition, the positions occupied by fragments of 6 consecutive amino acids to 1 amino acid less than any other full-length OBG3 polypeptide can also be envisaged based on these two examples and therefore are not individually listed solely for the purpose of not unnecessarily lengthening the specification. In preferred embodiments, OBG3 or gOBG3 polypeptide fragments having unexpected activity are selected from amino acids 84-244, 85-244, 86-244, 87-244, 88-244, 89-244, 90-244, 91-244, 92-244, 93-244, 94-244, 95-244, 96-244, 97-244, 98-244, 99-244, 100-244, 101-244, 102-244, or 103-244 of SEQ ID NO:6. In preferred embodiments, OBG3 or gOBG3 polypeptide fragments having unexpected activity are selected from amino acids 88-247, 89-247, 90-247, 91-247, 92-247, 93-247, 94-247, 95-247, 96-247, 97-247, 98-247, 99-247, 100-247, 101-247, 102-247, 103-247, 104-247, 105-247, or 106-247 of SEQ ID NO:2 or SEQ ID NO:4.

The OBG3 or gOBG3 polypeptide fragments of the present invention may alternatively be described by the formula “n to c” (inclusive); where “n” equals the N-terminal most amino acid position (as defined by the sequence listing) and “c” equals the C-terminal most amino acid position (as defined by the sequence listing) of the polypeptide; and further where “n” equals an integer between 1 and the number of amino acids of the full length polypeptide sequence of the present invention minus 6 (238 for SEQ ID NO: 6 and 241 for SEQ ID NOs: 2 or 4); and where “c” equals an integer between 7 and the number of amino acids of the full-length polypeptide sequence (244 for SEQ ID NO: 6 and 247 for SEQ ID NOs: 2 or 4); and where “n” is an integer smaller then “c” by at least 6. Therefore, for SEQ ID NO: 6, “n” is any integer selected from the list consisting of: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 234, 235, 236, 237 and 238; and “c” is any integer selected from the group consisting of: 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 234, 235, 236, 237, 238, 239, 240, 241, 242, 243, 244. Every combination of “n” and “c” positions are included as specific embodiments of the invention. Moreover, the formula “n” to “c” may be modified as ‘“n1-n2” to “c1-c2”’, wherein “n1-n2” and “c1-c2” represent positional ranges selected from any two integers above which represent amino acid positions of the sequence listing. Alternative formulas include ‘“n1-n2” to “c”’ and ‘“n” to “c1-c2”’. In preferred embodiment, OBG3 or gOBG3 polypeptide fragments of the invention may be described by the formula where n1=84, n2=103, and c=244 of SEQ ID NO:6 or by the formula n1=88, n2=106, and c=247 of SEQ ID NO:2 or SEQ D NO:4.

These specific embodiments, and other polypeptide and polynucleotide fragment embodiments described herein may be modified as being “at least”, “equal to”, “equal to or less than”, “less than”, “at least______but not greater than______” or “from______to______”. a specified size or specified N-terminal and/or C-terminal positions. It is noted that all ranges used to describe any embodiment of the present invention are inclusive unless specifically set forth otherwise.

The present invention also provides for the exclusion of any individual fragment specified by N-terminal and C-terminal positions or of any fragment specified by size in amino acid residues as described above. In addition, any number of fragments specified by N-terminal and C-terminal positions or by size in amino acid residues as described above may be excluded as individual species. Further, any number of fragments specified by N-terminal and C-terminal positions or by size in amino acid residues as described above may make up a polypeptide fragment in any combination and may optionally include non-OBG3 polypeptide sequence as well.

In particularly preferred embodiments, the OBG3 polypeptide fragment is a “globular OBG3” (gOBG3) fragment. The term “gOBG3 fragment” or “gOBG3” or “gOBG3 polypeptide” as used herein refers to fragments of a full-length OBG3 polypeptide that comprise at least 6 and any other integer number of amino acids up to 137 of the globular region of a full-length OBG3 polypeptide (defined above). In preferred embodiments, gOBG3 polypeptide fragments also comprise at least 1 and any other integer number of amino acids up 66 of the collagen region of a full-length OBG3 polypeptide, preferably 1,2,3,4,5,6,7,8,9,10,11,12,13,14,15,16,17,18,19,20,21,22, 23, or 24 consecutive amino acid residues from the collagen region of the intact OBG3 polypeptide that are adjacent to the globular region. By “adjacent” to the globular region is meant the first collagen amino acid immediately N-terminal to the globular region and adding each collagen amino acid consecutively in the N-terminal direction. Thus, for example, if there is only one collagen amino acid in the gOBG3 polypeptide fragment, it is the collagen amino acid 107 of SEQ ID NO:6 or amino acid 110 of SEQ ID NO:2 or SEQ ID NO:4 located adjacent and 5′ to the first amino acid of the globular region. If there are 24 collagen amino acids adjacent to the globular region in the gOBG3 fragment they would be the collagen amino acids 84-107 of SEQ ID NO:6 or amino acids 87-110 of SEQ ID NO:2 or SEQ ID NO:4.

In preferred embodiments, OBG3 or gOBG3 polypeptide fragments having unexpected activity are selected from amino acids 84-244, 85-244, 86-244, 87-244, 88-244, 89-244, 90-244, 91-244, 92-244, 93-244, 94-244, 95-244, 96-244, 97-244, 98-244, 99-244, 100-244, 101-244,102-244, or 103-244 of SEQ ID NO:6. In other preferred embodiments, OB03 or gOBG3 polypeptide fragments having unexpected activity are selected from amino acids 88-247, 89-247, 90-247, 91-247, 92-247, 93-247, 94-247, 95-247, 96-247, 97-247, 98-247, 99-247, 100-247, 101-247, 102-247, 103-247, 104-247, 105-247, or 106-247 of SEQ ID NO:2 or SEQ ID NO:4. In further preferred embodiments, gOBG3 polypeptide fragments are selected from amino acids 84 to 244, 85 to 244, 101 to 244, 102 to 244, or 103 to 244 of SEQ ID NO:6 and amino acids 88 to 247, 104 to 247, 105 to 247, or 106 to 247 of SEQ ID NO:2 or SEQ ID NO:4. In yet other preferred embodiments, the invention features a gOBG3 polypeptide fragment comprising at least 115, but not more than 175 contiguous amino acids of any one of the gOBG3 fragment sequences set forth in FIG. 1, wherein no more than 24 of said at least 115 and no more than 175 contiguous amino acids are present in the collagen-like region of OBG3. Preferably, the gOBG3 polypeptide fragment comprises at least 125, but not more than 165, or at least 140, but not more than 165 amino acids, and no more than 24 amino acids are in the collagen-like region; more preferably at least 125 but not more than 165, or at least 140 but not more than 165 amino acids, and no more than 12 amino acids are in the collagen-like region; or at least 140 and not more than 150 amino acids, and no more than 8 amino acids are present in the collagen-like region. Preferably the gOBG3 fragment is mammalian, preferably human or mouse, but most preferably human.

OBG3 and gOBG3 polypeptide fragments of the invention include variants, fragments, analogs and derivatives of the OBG3 and gOBG3 polypeptide fragments described above, including modified OBG3 and gOBG3 polypeptide fragments. Particularly preferred are proteolytically cleaved fragments of OBG3 of SEQ ID NO:6, SEQ ID NO:2, or SEQ ID NO:4. More preferred is OBG3 fragment of about amino acids 85-244 of SEQ ID NO:6 made by collagenase cleavage of SEQ ID NO:6 at about position 84. More preferred is OBG3 fragment of about amino acids 88-247 of SEQ ID NO:2 or of SEQ ID NO:4 made by collagenase cleavage of SEQ ID NO:2 or SEQ ID NO:4 at about position 87. More preferred is OBG3 fragment of about amino acids 85-244 of SEQ ID NO:6 made by matrix metalloproteinase-1 (MMP-1) cleavage of SEQ ID NO:6 at about position 84. More preferred is OBG3 fragment of about amino acids 88-247 of SEQ ID NO:2 or of SEQ ID NO:4 made by matrix metalloproteinase-1 (MMP-1) cleavage of SEQ ID NO:2 or SEQ ID NO:4 at about position 87. More preferred is OBG3 fragment of about amino acids 101-244 of SEQ ID NO:6 made by plasmin cleavage of SEQ ID NO:6 at about position 100. More preferred is OBG3 fragment of about amino acids 104-247 of SEQ ID NO:2 or of SEQ ID NO:4 made by plasmin cleavage of SEQ ID NO:2 or SEQ ID NO:4 at about position 103. More preferred is OBG3 fragment of about amino acids 103-244 of SEQ ID NO:6 made by precerebellin processing protease cleavage of SEQ ID NO:6 at about position 102. More preferred is OBG3 fragment of about amino acids 106-247 of SEQ ID NO:2 or of SEQ ID NO:4 made by precerebellin processing protease cleavage of SEQ ID NO:2 or SEQ ID NO:4 at about position 105. More preferred is APM1 proteolytic fragment of SEQ ID NO:6, wherein said APM1 fragment isolated from human plasma migrates with an apparent molecular weight of about 27 kDa on SDS-PAGE under reducing conditions. Most preferred are polypeptide fragments possessing biological activity, comprising amino acids 166-193, amino acids 166-176, and amino acids 167-176

Variants

It will be recognized by one of ordinary skill in the art that some amino acids of the OBG3 and gOBG3 fragment sequences of the present invention can be varied without significant effect on the structure or function of the protein; there will be critical amino acids in the fragment sequence that determine activity. Thus, the invention further includes variants of OBG3 and gOBG3 polypeptide fragments that have remyelinating activity as described above. Such variants include OBG3 fragment sequences with one or more amino acid deletions, insertions, inversions, repeats, and substitutions either from natural mutations or human manipulation selected according to general rules known in the art so as to have little effect on activity. Guidance concerning how to make phenotypically silent amino acid substitutions is provided below.

There are two main approaches for studying the tolerance of an amino acid sequence to change (see, Bowie, et al. (1990) Science, 247, 1306-10). The first method relies on the process of evolution, in which mutations are either accepted or rejected by natural selection. The second approach uses genetic engineering to introduce amino acid changes at specific positions of a cloned gene and selections or screens to identify sequences that maintain functionality.

These studies have revealed that proteins are surprisingly tolerant of amino acid substitutions and indicate which amino acid changes are likely to be permissive at a certain position of the protein. For example, most buried amino acid residues require nonpolar side chains, whereas few features of surface side chains are generally conserved. Other such phenotypically silent substitutions are described by Bowie et al. (supra) and the references cited therein.

Typically seen as conservative substitutions are the replacements, one for another, among the aliphatic amino acids Ala, Val, Leu and Phe; interchange of the hydroxyl residues Ser and Thr; exchange of the acidic residues Asp and Glu; substitution between the amide residues Asn and Gln; exchange of the basic residues Lys and Arg; and replacements among the aromatic residues Phe, Tyr. In addition, the following groups of amino acids generally represent equivalent changes: (1) Ala, Pro, Gly, Glu, Asp, Gln, Asn, Ser, Thr; (2) Cys, Ser, Tyr, Thr; (3) Val, Ile, Leu, Met, Ala, Phe; (4) Lys, Arg, His; (5) Phe, Tyr, Trp, His.

Similarly, amino acids in the OBG3 and gOBG3 polypeptide fragment sequences of the invention that are essential for function can also be identified by methods known in the art such as site-directed mutagenesis or alanine-scanning mutagenesis (see, e.g., Cunningham, et al. (1989) Science 244(4908):1081-5). The latter procedure introduces single alanine mutations at every residue in the molecule. The resulting mutant molecules are then tested for obesity-related activity using assays as described above. Of special interest are substitutions of charged amino acids with other charged or neutral amino acids that may produce proteins with highly desirable improved characteristics, such as less aggregation. Aggregation may not only reduce activity but also be problematic when preparing pharmaceutical or physiologically acceptable formulations, because aggregates can be immunogenic (see, e.g., Pinckard, et al., (1967) Clin. Exp. Immunol 2:331-340; Robbins, et al., (1987) Diabetes Jul;36(7):838-41; and Cleland, et al., (1993) Crit Rev Ther Drug Carrier Syst. 10(4):307-77).

Thus, the fragment, derivative, analog, or homolog of the OBG3 or gOBG3 fragment of the present invention may be, for example: (i) one in which one or more of the amino acid residues are substituted with a conserved or non-conserved amino acid residue (preferably a conserved amino acid residue) and such substituted amino acid residue may or may not be one encoded by the genetic code (i.e. may be a non-naturally occurring amino acid); or (ii) one in which one or more of the amino acid residues includes a substituent group; or (iii) one in which the OBG3 or gOBG3 fragment is fused with another compound, such as a compound to increase the half-life of the fragment (for example, polyethylene glycol); or (iv) one in which the additional amino acids are fused to the above form of the fragment, such as an IgG Fc fusion region peptide or leader or secretory sequence or a sequence which is employed for purification of the above form of the fragment or a pro-protein sequence. Such fragments, derivatives and analogs are deemed to be within the scope of those skilled in the art from the teachings herein.

A further embodiment of the invention relates to a polypeptide which comprises the amino acid sequence of an OBG3 or gOBG3 polypeptide fragment having an amino acid sequence which contains at least one conservative amino acid substitution, but not more than 50 conservative amino acid substitutions, not more than 40 conservative amino acid substitutions, not more than 30 conservative amino acid substitutions, and not more than 20 conservative amino acid substitutions. Also provided are polypeptides which comprise the amino acid sequence of a OBG3 or gOBG3 fragment, having at least one, but not more than 10, 9, 8, 7, 6, 5, 4, 3, 2 or 1 conservative amino acid substitutions.

Another specific embodiment of a modified OBG3 or gOBG3 fragment of the invention is a polypeptide that is resistant to proteolysis, for example a OBG3 or gOBG3 fragment in which a —CONH— peptide bond is modified and replaced by one or more of the following: a (CH2NH) reduced bond; a (NHCO) retro inverso bond; a (CH2-O) methylene-oxy bond; a (CH2-S) thiomethylene bond; a (CH2CH2) carba bond; a (CO—CH2) cetomethylene bond; a (CHOH—CH2) hydroxyethylene bond); a (N—N) bound; a E-alcene bond; or a —CH═CH— bond. Thus, the invention also encompasses an OBG3 or gOBG3 fragment or a variant thereof in which at least one peptide bond has been modified as described above.

A further embodiment of the invention relates to an OBG3 or gOBG3 polypeptide fragment made resistant to dipeptidyl peptidase cleavage through N-terminal modification of said polypeptide fragment. In preferred embodiment, said OBG3 or gOBG3 polypeptide fragment is selected from amino acids 85-244 or 103-244 of SEQ ID NO:6 or amino acids 106-247 of SEQ ID NO:2 or SEQ ID NO:4. In preferred embodiment, said dipeptidyl peptidase cleavage leads to removal of the N-terminal dipeptide EP by dipeptidyl peptidase from said preferred gOBG3 polypeptide fragment 103-244 of SEQ ID NO:6 or 106-247 of SEQ ID NO:2 or SEQ ID NO:4. In preferred embodiment, said dipeptidyl peptidase cleavage leads to removal of the N-terminal dipeptide VP by dipeptidyl peptidase from said preferred gOBG3 polypeptide fragment 85-244 of SEQ ID NO:6. In preferred embodiment, said dipeptidyl peptidase is human plasma comprised of dipeptidyl peptidase. In preferred embodiment, said dipeptidyl peptidase is selected from but not restricted to human CD26 or human Attractin. In further preferred embodiment, said dipeptidyl peptidase is selected from soluble human CD26 or soluble human Attractin. In preferred embodiment, said N-terminal modification is selected from but not restricted to glycation [Harte (2001) Regulatory Peptides 96:95-104 which disclosure is hereby incorporated by reference in its entirety], N-methylation, alpha-methylation, desamidation [Gallwitz (2000) Regulatory Peptides 86:103-111 which disclosure is hereby incorporated by reference in its entirety], or alternation of the chirality of one or more N-terminal amino acids [Siegel (1999) European Journal of Clinical Investigation 29:610-614 which disclosure is hereby incorporated by reference in its entirety]. Thus, the invention also encompasses an OBG3 or gOBG3 polypeptide fragment or a variant thereof that has been made resistant to dipeptidyl peptidase cleavage through N-terminal modification of said polypeptide fragment.

In addition, amino acids have chirality within the body of either L or D. In some embodiments it is preferable to alter the chirality of the amino acids in the OBG3 or gOBG3 polypeptide fragments of the invention in order to extend half-life within the body. In other embodiments, it is preferable to alter the chirality of one or more amino acid in order to render the OBG3 or gOBG3 polypeptide fragment resistant to dipeptidyl peptidase cleavage [Siegel (1999) European Journal of Clinical Investigation 29:610-614 which disclosure is hereby incorporated by reference in its entirety]. In further embodiments, it is preferable to alter the chirality of the penultimate N-terminal amino acid in order to render the OBG3 or gOBG3 polypeptide fragment resistant to dipeptidyl peptidase cleavage. Thus, in some embodiments, one or more of the amino acids are preferably in the L configuration. In other embodiments, one or more of the amino acids are preferably in the D configuration.

Percent Identity

The polypeptides of the present invention also include polypeptides having an amino acid sequence at least 50% identical, at least 60% identical, or 70%, 80%, 85%, 90%, 91%, 92%, 93%, 94%, 95%, 96%, 97%, 98% or 99% identical to an OBG3 or gOBG3 fragment as described above. By a polypeptide having an amino acid sequence at least, for example, 95% “identical” to an OBG3 or gOBG3 fragment amino acid sequence is meant that the amino acid sequence is identical to the OBG3 or gOBG3 polypeptide fragment sequence except that it may include up to five amino acid alterations per each 100 amino acids of the OBG3 or gOBG3 polypeptide fragment amino acid sequence. The reference sequence is the OBG3 or gOBG3 polypeptide fragment with a sequence corresponding to the sequence of the sequence listing. Thus, to obtain a polypeptide having an amino acid sequence at least 95% identical to an OBG3 or gOBG3 fragment amino acid sequence, up to 5% (5 of 100) of the amino acid residues in the sequence may be inserted, deleted, or substituted with another amino acid compared with the OBG3 or gOBG3 polypeptide fragment sequence. These alterations may occur at the amino or carboxy termini or anywhere between those terminal positions, interspersed either individually among residues in the sequence or in one or more contiguous groups within the sequence.

As a practical matter, whether any particular polypeptide is a percentage identical to an OBG3 or gOBG3 fragment can be determined conventionally using known computer programs. Such algorithms and programs include, but are by no means limited to, TBLASTN, BLASTP, FASTA, TFASTA, and CLUSTALW (Pearson and Lipman, (1988) Proc Natl Acad Sci USA 85(8):2444-8; Altschul et al., (1990) J Mol Biol 215(3):403-410; Thompson et al., (1994) Nucleic Acids Res 22(2):4673-4680; Higgins et al., (1996) Meth Enzymol 266:383-402; Altschul et al., (1997) Nuc Acids Res 25:3389-3402; Altschul et al., (1993) Nature Genetics 3:266-272). In a particularly preferred embodiment, protein and nucleic acid sequence homologies are evaluated using the Basic Local Alignment Search Tool (“BLAST”), which is well known in the art (See, e.g., Karlin and Altschul (1990) Proc Natl Acad Sci USA 87(6):2264-8; Altschul et al., 1990, 1993, 1997, all supra). In particular, five specific BLAST programs are used to perform the following tasks:
(1) BLASTP and BLAST3 compare an amino acid query sequence against a protein sequence database;
(2) BLASTN compares a nucleotide query sequence against a nucleotide sequence database;
(3) BLASTX compares the six-frame conceptual translation products of a query nucleotide sequence (both strands) against a protein sequence database;
(4) TBLASTN compares a query protein sequence against a nucleotide sequence database translated in all six reading frames (both strands); and
(5) TBLASTX compares the six-frame translations of a nucleotide query sequence against the six-frame translations of a nucleotide sequence database.

The BLAST programs identify homologous sequences by identifying similar segments, which are referred to herein as “high-scoring segment pairs,” between a query amino or nucleic acid sequence and a test sequence which is preferably obtained from a protein or nucleic acid sequence database. High-scoring segment pairs are preferably identified (i.e., aligned) by means of a scoring matrix, many of which are known in the art. Preferably, the scoring matrix used is the BLOSUM62 matrix (see, Gonnet et al., (1992) Science 256(5062):1443-5; Henikoff and Henikoff (1993) Proteins 17(1):49-61). Less preferably, the PAM or PAM250 matrices may also be used (See, e.g. Schwartz and Dayhoff, eds, (1978) Matrices for Detecting Distance Relationships: Atlas of Protein Sequence and Structure, Washington: National Biomedical Research Foundation). The BLAST programs evaluate the statistical significance of all high-scoring segment pairs identified, and preferably selects those segments which satisfy a user-specified threshold of significance, such as a user-specified percent homology. Preferably, the statistical significance of a high-scoring segment pair is evaluated using the statistical significance formula of Karlin (See, e.g., Karlin and Altschul, (1990) Proc Natl Acad Sci USA 87(6):2264-8). The BLAST programs may be used with the default parameters or with modified parameters provided by the user. Preferably, the parameters are default parameters.

A preferred method for determining the best overall match between a query sequence (a sequence of the present invention) and a subject sequence, also referred to as a global sequence alignment, can be determined using the FASTDB computer program based on the algorithm of Brutlag et al. (1990) Comp. App. Biosci. 6:237-245. In a sequence alignment the query and subject sequences are both amino acid sequences. The result of said global sequence alignment is in percent identity. Preferred parameters used in a FASTDB amino acid alignment are: Matrix-PAM 0, k-tuple=2, Mismatch Penalty=1, Joining Penalty=20, Randomization Group=25 Length=0, Cutoff Score=1, Window Size=sequence length, Gap Penalty=5, Gap Size Penalty=0.05, Window Size=247 or the length of the subject amino acid sequence, whichever is shorter.

If the subject sequence is shorter than the query sequence due to N-or C-terminal deletions, not because of internal deletions, the results, in percent identity, must be manually corrected because the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C- terminal of the subject sequence, that are not matched/aligned with a corresponding subject residue, as a percent of the total bases of the query sequence. Whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present invention. Only residues to the N- and C-termini of the subject sequence, which are not matched/aligned with the query sequence, are considered for the purposes of manually adjusting the percent identity score. That is, only query amino acid residues outside the farthest N- and C-terminal residues of the subject sequence.

For example, a 90 amino acid residue subject sequence is aligned with a 100-residue query sequence to determine percent identity. The deletion occurs at the N-terminus of the subject sequence and therefore, the FASTDB alignment does not match/align with the first residues at the N-terminus. The 10 unpaired residues represent 10% of the sequence (number of residues at the N- and C- termini not matched/total number of residues in the query sequence) so 10% is subtracted from the percent identity score calculated by the FASTDB program. If the remaining 90 residues were perfectly matched the final percent identity would be 90%.

In another example, a 90-residue subject sequence is compared with a 100-residue query sequence. This time the deletions are internal so there are no residues at the N- or C-termini of the subject sequence, which are not matched/aligned with the query. In this case, the percent identity calculated by FASTDB is not manually corrected. Once again, only residue positions outside the N- and C-terminal ends of the subject sequence, as displayed in the FASTDB alignment, which are not matched/aligned with the query sequence are manually corrected. No other manual corrections are made for the purposes of the present invention.

Production

Note, throughout the disclosure, wherever OBG3 polypeptide fragments are discussed, gOBG3fragments are specifically intended to be included as a preferred subset of OBG3 polypeptide fragments.

OBG3 polypeptide fragments are preferably isolated from human or mammalian tissue samples or expressed from human or mammalian genes in human or mammalian cells. The OBG3 polypeptide fragments of the invention can be made using routine expression methods known in the art. The polynucleotide encoding the desired polypeptide fragments is ligated into an expression vector suitable for any convenient host. Both eukaryotic and prolcaryotic host systems are used in forming recombinant polypeptide fragments. The polypeptide fragment is then isolated from lysed cells or from the culture medium and purified to the extent needed for its intended use. Purification is by any technique known in the art, for example, differential extraction, salt fractionation, chromatography, centrifugation, and the like. See, for example, Methods in Enzymology for a variety of methods for purifying proteins. Also, see Examples 1-3 for methods previously used for OBG3 polypeptide fragments.

In an alternative embodiment, the polypeptides of the invention are isolated from milk. The polypeptides can be purified as full-length OBG3 polypeptides, which can then be cleaved, if appropriate, in vitro to generate an OBG3 fragment, or, alternatively, OBG3 fragments themselves can be purified from the milk. Any of a large number of methods can be used to purify the present polypeptides from milk, including those taught in Protein Purification Applications, A Practical Approach (New Edition), Edited by Simon Roe, AEA Technology Products and Systems, Biosciences, Harwell; Clark (1998) J Mammary Gland Biol Neoplasia 3:337-50; Wilkins and Velander (1992) 49:333-8; U.S. Pat. Nos. 6,140,552; 6,025,540; Hennighausen, Protein Expression and Purification, vol. 1, pp. 3-8 (1990); Harris et al. (1997) Bioseparation 7:31-7; Degener et al. (1998) J Chromatog 799:125-37; Wilkins (1993) J Cell Biochem Suppl. 0 (17 part A):39; the entire disclosures of each of which are herein incorporated by reference. In a typical embodiment, milk is centrifuged, e.g. at a relatively low speed, to separate the lipid fraction, and the aqueous supernatant is then centrifuged at a higher speed to separate the casein in the milk from the remaining, “whey” fraction. Often, biomedical proteins are found in this whey fraction, and can be isolated from this fraction using standard chromatographic or other procedures commonly used for protein purification, e.g. as described elsewhere in the present application. In one preferred embodiment, OBG3 polypeptides are purified using antibodies specific to OBG3 polypeptides, e.g. using affinity chromatography. In addition, methods can be used to isolate particular OBG3 fragments, e.g. electrophoretic or other methods for isolating proteins of a particular size. The OBG3 polypeptides isolating using these methods can be naturally occurring, as OBG3 polypeptides have been discovered to be naturally present in the milk of mammals (see, e.g. Example 17), or can be the result of the recombinant production of the protein in the mammary glands of a non-human mammal, as described infra. In one such embodiment, the OBG3 fragment is produced as a fusion protein with a heterologous, antigenic polypeptide sequence, which antigenic sequence can be used to purify the protein, e.g., using standard immuno-affinity methodology.

In addition, shorter protein fragments may be produced by chemical synthesis. Alternatively, the proteins of the invention are extracted from cells or tissues of humans or non-human animals. Methods for purifying proteins are known in the art, and include the use of detergents or chaotropic agents to disrupt particles followed by differential extraction and separation of the polypeptides by ion exchange chromatography, affinity chromatography, sedimentation according to density, and gel electrophoresis.

Any OBG3 fragment cDNA, including that in FIG. 4, can be used to express OBG3 polypeptide fragments. The nucleic acid encoding the OBG3 fragment to be expressed is operably linked to a promoter in an expression vector using conventional cloning technology. The OBG3 fragment cDNA insert in the expression vector may comprise the coding sequence for: the full-length OBG3 polypeptide (to be later modified); from 6 amino acids to 6 amino acids less than the full-length OBG3 polypeptide; a gOBG3 fragment; or variants and % similar polypeptides.

The expression vector is any of the mammalian, yeast, insect or bacterial expression systems known in the art, some of which are described herein, and examples of which are given in the Examples (Examples 1-3). Commercially available vectors and expression systems are available from a variety of suppliers including Genetics Institute (Cambridge, Mass.), Stratagene (La Jolla, Calif.), Promega (Madison, Wis.), and Invitrogen (San Diego, Calif.). If desired, to enhance expression and facilitate proper protein folding, the codon context and codon pairing of the sequence can be optimized for the particular expression organism into which the expression vector is introduced, as explained by Hatfield, et al., U.S. Pat. No. 5,082,767, the disclosures of which are incorporated by reference herein in their entirety.

If the nucleic acid encoding OBG3 polypeptide fragments lacks a methionine to serve as the initiation site, an initiating methionine can be introduced next to the first codon of the nucleic acid using conventional techniques. Similarly, if the insert from the OBG3 polypeptide fragment cDNA lacks a poly A signal, this sequence can be added to the construct by, for example, splicing out the Poly A signal from pSG5 (Stratagene) using BglI and SalI restriction endonuclease enzymes and incorporating it into the mammalian expression vector pXT1 (Stratagene). pXT1 contains the LTRs and a portion of the gag gene from Moloney Murine Leulemia Virus. The position of the LTRs in the construct allow efficient stable transfection. The vector includes the Herpes Simplex Thymidine Kinase promoter and the selectable neomycin gene.

The nucleic acid encoding an OBG3 fragment can be obtained by PCR from a vector containing the OBG3 nucleotide sequence using oligonucleotide primers complementary to the desired OBG3 cDNA and containing restriction endonuclease sequences for Pst I incorporated into the 5′ primer and BglII at the 5′ end of the corresponding cDNA 3′ primer, taking care to ensure that the sequence encoding the OBG3 fragment is positioned properly with respect to the poly A signal. The purified fragment obtained from the resulting PCR reaction is digested with PstI, blunt ended with an exonuclease, digested with Bgl II, purified and ligated to pXT1, now containing a poly A signal and digested with BglII. Alternative methods are presented in Examples 1-3.

Transfection of an OBG3 fragment-expressing vector into mouse NIH 3T3 cells is one embodiment of introducing polynucleotides into host cells. Introduction of a polynucleotide encoding a polypeptide into a host cell can be effected by calcium phosphate transfection, DEAE-dextran mediated transfection, cationic lipid-mediated transfection, electroporation, transduction, infection, or other methods. Such methods are described in many standard laboratory manuals, such as Davis et al. ((1986) Methods in Molecular Biology, Elsevier Science Publishing Co., Inc., Amsterdam). It is specifically contemplated that the polypeptides of the present invention may in fact be expressed by a host cell lacking a recombinant vector. Methods of expressing OBG3 fragment of the invention in cells are described in Examples 1-3.

A polypeptide of this invention (i.e. an OBG3 or gOBG3 fragment) can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography (“HPLC”) is employed for purification. Polypeptides of the present invention, and preferably the secreted form, can also be recovered from: products purified from natural sources, including bodily fluids, tissues and cells, whether directly isolated or cultured; products of chemical synthetic procedures; and products produced by recombinant techniques from a prokaryotic or eukaryotic host, including, for example, bacterial, yeast, higher plant, insect, and mammalian cells.

Depending upon the host employed in a recombinant production procedure, the polypeptides of the present invention may be glycosylated or may be non-glycosylated. Preferably the polypeptides of the invention are non-glycosylated. In addition, polypeptides of the invention may also include an initial modified methionine residue, in some cases as a result of host-mediated processes. Thus, it is well known in the art that the N-terminal methionine encoded by the translation initiation codon generally is removed with high efficiency from any protein after translation in all eukaryotic cells. While the N-terminal methionine on most proteins also is efficiently removed in most prokaryotes, for some proteins, this prokaryotic removal process is inefficient, depending on the nature of the amino acid to which the N-terminal methionine is covalently linked.

In addition to encompassing host cells containing the vector constructs discussed herein, the invention also encompasses primary, secondary, and immortalized host cells of vertebrate origin, particularly mammalian origin, that have been engineered to delete or replace endogenous genetic material (e.g., coding sequence), and/or to include genetic material (e.g., heterologous polynucleotide sequences) that is operably associated with the polynucleotides of the invention, and which activates, alters, and/or amplifies endogenous polynucleotides. For example, techniques known in the art may be used to operably associate heterologous control regions (e.g., promoter and/or enhancer) and endogenous polynucleotide sequences via homologous recombination, see, e.g., U.S. Pat. No. 5,641,670, issued Jun. 24, 1997; International Publication No. WO 96/29411, published Sep. 26, 1996; International Publication No. WO 94/12650, published Aug. 4, 1994; Koller et al., (1989) Proc Natl Acad Sci USA 86(22):8932-5; Koller et al., (1989) Proc Natl Acad Sci USA 86(22):8927-31; and Zijlstra et al. (1989) Nature 342(6248):435-8; the disclosures of each of which are incorporated by reference in their entireties).

Modifications

In addition, polypeptides of the invention can be chemically synthesized using techniques known in the art (See, e.g., Creighton, 1983 Proteins. New York, N.Y.: W. H. Freeman and Company; and Hunkapiller et al., (1984) Nature 310(5973):105-11). For example, a relative short fragment of the invention can be synthesized by use of a peptide synthesizer. Furthermore, if desired, nonclassical amino acids or chemical amino acid analogs can be introduced as a substitution or addition into the fragment sequence. Non-classical amino acids include, but are not limited to, to the D-isomers of the common amino acids, 2,4-diaminobutyric acid, a-amino isobutyric acid, 4-aminobutyric acid, Abu, 2-amino butyric acid, g-Abu, e-Ahx, 6-amino hexanoic acid, Alb, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, b-alanine, fluoroamino acids, designer amino acids such as b-methyl amino acids, Ca-methyl amino acids, Na-methyl amino acids, and amino acid analogs in general. Furthermore, the amino acid can be D (dextrorotary) or L (levorotary).

The invention encompasses polypeptide fragments which are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited, to specific chemical cleavage by cyanogen bromide, typsin, chymotrypsin, papain, V8 protease, NaBH4; acetylation, formylation, oxidation, reduction; metabolic synthesis in the presence of tunicamycin; etc.

Additional post-translational modifications encompassed by the invention include, for example, N-linked or 0-linked carbohydrate chains, processing of N-terminal or C-terminal ends), attachment of chemical moieties to the amino acid backbone, chemical modifications of N-linked or O-linked carbohydrate chains, and addition or deletion of an N-terminal methionine residue as a result of procaryotic host cell expression. The polypeptide fragments may also be modified with a detectable label, such as an enzymatic, fluorescent, isotopic or affinity label to allow for detection and isolation of the polypeptide.

Also provided by the invention are chemically modified derivatives of the polypeptides of the invention that may provide additional advantages such as increased solubility, stability and circulating time of the polypeptide, or decreased immunogenicity. See U.S. Pat. No: 4,179,337. The chemical moieties for derivitization may be selected from water soluble polymers such as polyethylene glycol, ethylene glycol/propylene glycol copolymers, carboxymethylcellulose, dextran, polyvinyl alcohol and the like. The polypeptides may be modified at random positions within the molecule, or at predetermined positions within the molecule and may include one, two, three or more attached chemical moieties.

The polymer may be of any molecular weight, and may be branched or unbranched. For polyethylene glycol, the preferred molecular weight is between about 1 kDa and about 100 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on biological activity, the ease in handling, the degree or lack of antigenicity and other known effects of the polyethylene glycol to a therapeutic protein or analog).

The polyethylene glycol molecules (or other chemical moieties) should be attached to the polypeptide with consideration of effects on functional or antigenic domains of the polypeptide. There are a number of attachment methods available to those skilled in the art, e.g., EP 0 401 384, herein incorporated by reference (coupling PEG to G-CSF), see also Malik et al. (1992) Exp Hematol 20(8):1028-35, reporting pegylation of GM-CSF using tresyl chloride). For example, polyethylene glycol may be covalently bound through amino acid residues via a reactive group, such as, a free amino or carboxyl group. Reactive groups are those to which an activated polyethylene glycol molecule may be bound. The amino acid residues having a free amino group may include lysine residues and the N-terminal amino acid residues; those having a free carboxyl group may include aspartic acid residues, glutamic acid residues and the C-terminal amino acid residue. Sulfhydryl groups may also be used as a reactive group for attaching the polyethylene glycol molecules. Preferred for therapeutic purposes is attachment at an amino group, such as attachment at the N-terminus or lysine group.

One may specifically desire proteins chemically modified at the N-terminus. Using polyethylene glycol as an illustration of the present composition, one may select from a variety of polyethylene glycol molecules (by molecular weight, branching, etc.), the proportion of polyethylene glycol molecules to protein (polypeptide) molecules in the reaction mix, the type of pegylation reaction to be performed, and the method of obtaining the selected N-terminally pegylated protein. The method of obtaining the N-terminally pegylated preparation (i.e., separating this moiety from other monopegylated moieties if necessary) may be by purification of the N-terminally pegylated material from a population of pegylated protein molecules. Selective proteins chemically modified at the N-terminus may be accomplished by reductive alkylation, which exploits differential reactivity of different types of primary amino groups (lysine versus the N-terminal) available for derivatization in a particular protein. Under the appropriate reaction conditions, substantially selective derivatization of the protein at the N-terminus with a carbonyl group containing polymer is achieved.

In further preferred embodiment, the invention features a method of reducing body mass comprising providing or administering to individuals in need of reducing body mass said pharmaceutical or physiologically acceptable composition described in the fifth aspect in combination with provision or administration of an antagonist of dipeptidyl peptidase cleavage of OBG3 or gOBG3 polypeptide fragment of the first aspect.

Preferred said antagonist is a peptidyl derivative of a diester of alpha-aminoalkylphosphonic acid (U.S. Pat. No. 5,543,396 which disclosure is hereby incorporated by reference in its entirety). More preferred said peptidyl derivative is selected from Ala-Pro^{P}(OZ)₂, AcOH.Ala-Pip^{P}(Oph)₂, HCl.Ala-Pro^{P}(Oph-4Cl)₂, HCl.Ala-Pip^{P}(Oph-4Cl)₂, or 2HCl.Lys-Pip^{P}(Oph-4Cl)₂, where Z represents an aryl group, a substituted aryl group or a highly flourinated alkyl group, Pro^{P} represents a proline phosphonate derivative, and Pip^{P} represents piperidyl phosphonate (U.S. Pat. No. 5,543,396 which disclosure is hereby incorporated by reference in its entirety).

Other preferred said antagonist is a compound of the general formula Z-Xaa-Y′, in which Xaa is an amino acid, Z is a protecting group, and Y′ is one of various types of ring structures (U.S. Pat. No. 6,090,786 which disclosure is hereby incorporated by reference in its entirety). More preferred is said compound wherein Z may or may not be present and represents a protecting group, such as benzyloxycarbonyl; Xaa represents alanine, methionine, arginine, phenylalanine, aspartic acid, proline, asparagine, serine, cysteine, threonine, glycine, tyrosine, glutamic acid, tryptophan, glutamine, valine, isoleucine, lysine, leucine, L-thioproline, L-homoproline, L-1,2,3,4,tetrahydroisoquinoline-3-carboxylic acid (Tic), L-2,3-dihydroindol-2-carboxylic acid, L-naphthylglycine, L-phenylglycine, L-4-phenylproline, O-benzyl tyrosine, omega-Z lysine, or omega-acetyl lysine; and Y′ represents a pyrrolidide, a phosphonate or phosphinate derivative, or reduced peptide; or pharmaceutically acceptable salts thereof (U.S. Pat. No. 6,090,786 which disclosure is hereby incorporated by reference in its entirety).

Other preferred said antagonist is sulphostin (U.S. Pat. No. 6,214,340 which disclosure is hereby incorporated by reference in its entirety).

Other preferred said antagonist is N-(substituted glycyl)-2-cyanopyrrolidine (U.S. Pat. No. 6,166,063). More preferred said N-(substituted glycyl)-2-cyanopyrrolidine is selected from pyrrolidine, 1-[[(3,5-dimethyl-1-adamantyl)amino]-acetyl]-2-cyano-, (S)—; pyrrolidine, 1-[[(3-ethyl-1-adamantyl)amino]-acetyl]-2-cyano-, (S)—; pyrrolidine, 1-[[(3-methoxy-1-adamantyl)amino]-acetyl]-2-cyano-, (S)—; pyrrolidine, 1-[[[3-[[(t-butylamino)carbonyl]oxy]-1-adamantyl]amino]-acetyl]-2-cyano-, (S)—; pyrrolidine, 1-[[[3-[[[(4-methoxyphenyl)amino]-carbonyl]oxy]-1-adamantyl]amino]-acetyl]-2-cyano-, (S)—; pyrrolidine, 1-[[[(3-[[(phenylamino)carbonyl]oxy]-1-adamantyl]amino]-acetyl]-2-cyano-, (S)—; pyrrolidine, 1-[[(5-hydroxy-2-adamantyl))amino]-acetyl]-2-cyano-, (S)—; pyrrolidine, 1-[[(3-acetyloxy-1-adamantyl)amino]-acetyl]-2-cyano-, (S)—; pyrrolidine, 1-[[[(3-[[[(diisopropyl)amino]carbonyl]oxy]-1-adamantyl]amino]-acetyl]-2-cyano-, (S)—; pyrrolidine, 1-[[[3-[[[(cyclohexyl)amino]carbonyl]oxy]-1-adamantyl]amino]-acetyl]-2-cyano-, (S)—; pyrrolidine, 1-[[(3-ethyoxy-1-adamantyl)amino]-acetyl)-2-cyano-, (S)—; or, in each case, a pharmaceutically acceptable acid addition salt thereof (U.S. Pat. No. 6,166,063).

Other preferred said antagonist is tetrahydroisoquinohne 3-carboxamide derivative of formula ##STR1## (U.S. Pat. No. 6,172,081). More preferred is said derivative and pharmaceutically acceptable salts thereof wherein X is CH₂, S, O, or C(CH₃)₂; R₁ and R₂ are independently hydrogen, hydroxy, alkyl, alkoxy, aralkoxy, or halogen (U.S. Pat. No. 6,172,081).

Other preferred said antagonist is valine-pyrrolidide [Deacon (2001) Diabetes 50:1588-1597 which disclosure is hereby incorporated by reference in its entirety].

Multimers

The polypeptide fragments of the invention may be in monomers or multimers (i.e., dimers, trimers, tetramers and higher multimers). Accordingly, the present invention relates to monomers and multimers of the polypeptide fragments of the invention, their preparation, and compositions (preferably, pharmaceutical or physiologically acceptable compositions) containing them. In specific embodiments, the polypeptides of the invention are monomers, dimers, trimers or tetramers. In additional embodiments, the multimers of the invention are at least dimers, at least trimers, or at least tetramers.

Multimers encompassed by the invention may be homomers or heteromers. As used herein, the term homomer, refers to a multimer containing only polypeptides corresponding to the OBG3 polypeptide fragments of the invention (including polypeptide fragments, variants, splice variants, and fusion proteins corresponding to these polypeptide fragments as described herein). These homomers may contain polypeptide fragments having identical or different amino acid sequences. In a specific embodiment, a homomer of the invention is a multimer containing only polypeptide fragments having an identical amino acid sequence. In another specific embodiment, a homomer of the invention is a multimer containing polypeptide fragments having different amino acid sequences. In specific embodiments, the multimer of the invention is a homodimer (e.g., containing polypeptide fragments having identical or different amino acid sequences) or a homotrimer (e.g., containing polypeptide fragments having identical and/or different amino acid sequences). In additional embodiments, the homomeric multimer of the invention is at least a homodimer, at least a homotrimer, or at least a homotetramer.

As used herein, the term heteromer refers to a multimer containing one or more heterologous polypeptides (i.e., corresponding to different proteins or polypeptide fragments thereof) in addition to the polypeptides of the invention. In a specific embodiment, the multimer of the invention is a heterodimer, a heterotrimer, or a heterotetramer. In additional embodiments, the heteromeric multimer of the invention is at least a heterodimer, at least a heterotrimer, or at least a heterotetramer.

Multimers of the invention may be the result of hydrophobic, hydrophilic, ionic and/or covalent associations and/or may be indirectly linked, by for example, liposome formation. Thus, in one embodiment, multimers of the invention, such as, for example, homodimers or homotrimers, are formed when polypeptides of the invention contact one another in solution. In another embodiment, heteromultimers of the invention, such as, for example, heterotrimers or heterotetramers, are formed when polypeptides of the invention contact antibodies to the polypeptides of the invention (including antibodies to the heterologous polypeptide sequence in a fusion protein of the invention) in solution. In other embodiments, multimers of the invention are formed by covalent associations with and/or between the polypeptides of the invention. Such covalent associations may involve one or more amino acid residues contained in the polypeptide sequence (e.g., that recited in the sequence listing, or contained in the polypeptide encoded by a deposited clone). In one instance, the covalent associations are cross-linking between cysteine residues located within the polypeptide sequences, which interact in the native (i.e., naturally occurring) polypeptide. In another instance, the covalent associations are the consequence of chemical or recombinant manipulation. Alternatively, such covalent associations may involve one or more amino acid residues contained in the heterologous polypeptide sequence in a fusion protein of the invention.

In one example, covalent associations are between the heterologous sequence contained in a fusion protein of the invention (see, e.g., U.S. Pat. No. 5,478,925). In a specific example, the covalent associations are between the heterologous sequence contained in an Fc fusion protein of the invention (as described herein). In another specific example, covalent associations of fusion proteins of the invention are between heterologous polypeptide sequence from another protein that is capable of forming covalently associated multimers, such as for example, oseteoprotegerin (see, e.g., International Publication NO: WO 98/49305, the contents of which are herein incorporated by reference in its entirety). In another embodiment, two or more polypeptides of the invention are joined through peptide linkers. Examples include those peptide linkers described in U.S. Pat. No. 5,073,627 (hereby incorporated by reference). Proteins comprising multiple polypeptides of the invention separated by peptide linkers may be produced using conventional recombinant DNA technology.

Another method for preparing multimer polypeptides of the invention involves use of polypeptides of the invention fused to a leucine zipper or isoleucine zipper polypeptide sequence. Leucine zipper and isoleucine zipper domains are polypeptides that promote multimerization of the proteins in which they are found. Leucine zippers were originally identified in several DNA-binding proteins, and have since been found in a variety of different proteins (Landschulz et al., (1988) Genes Dev. Jul;2(7):786-800). Among the known leucine zippers are naturally occurring peptides and derivatives thereof that dimerize or trimerize. Examples of leucine zipper domains suitable for producing soluble multimeric proteins of the invention are those described in PCT application WO 94/10308, hereby incorporated by reference. Recombinant fusion proteins comprising a polypeptide of the invention fused to a polypeptide sequence that dimerizes or trimerizes in solution are expressed in suitable host cells, and the resulting soluble multimeric fusion protein is recovered from the culture supernatant using techniques known in the art.

Trimeric polypeptides of the invention may offer the advantage of enhanced biological activity. Preferred leucine zipper moieties and isoleucine moieties are those that preferentially form trimers. One example is a leucine zipper derived from lung surfactant protein D (SPD), as described in Hoppe et al. FEBS Letters (1994) 344(2-3):191-5 and in U.S. patent application Ser. No. 08/446,922, hereby incorporated by reference. Other peptides derived from naturally occurring trimeric proteins may be employed in preparing trimeric polypeptides of the invention. In another example, proteins of the invention are associated by interactions between Flag® & polypeptide sequence contained in fusion proteins of the invention containing Flag® polypeptide sequence. In a further embodiment, proteins of the invention are associated by interactions between heterologous polypeptide sequence contained in Flag® fusion proteins of the invention and anti Flag® antibody.

The multimers of the invention may be generated using chemical techniques known in the art. For example, polypeptides desired to be contained in the multimers of the invention may be chemically cross-linked using linker molecules and linker molecule length optimization techniques known in the art (see, e.g., U.S. Pat. No. 5,478,925, which is herein incorporated by reference in its entirety). Additionally, multimers of the invention may be generated using techniques known in the art to form one or more inter-molecule cross-links between the cysteine residues located within the sequence of the polypeptides desired to be contained in the multimer (see, e.g., U.S. Pat. No. 5,478,925, which is herein incorporated by reference in its entirety). Further, polypeptides of the invention may be routinely modified by the addition of cysteine or biotin to the C-terminus or N-terminus of the polypeptide and techniques known in the art may be applied to generate multimers containing one or more of these modified polypeptides (see, e.g., U.S. Pat. No. 5,478,925, which is herein incorporated by reference in its entirety). Additionally, at least 30 techniques known in the art may be applied to generate liposomes containing the polypeptide components desired to be contained in the multimer of the invention (see, e.g., U.S. Pat. No. 5,478,925, which is herein incorporated by reference in its entirety).

Alternatively, multimers of the invention may be generated using genetic engineering techniques known in the art. In one embodiment, polypeptides contained in multimers of the invention are produced recombinantly using fusion protein technology described herein or otherwise known in the art (see, e.g., U.S. Pat. No. 5,478,925, which is herein incorporated by reference in its entirety). In a specific embodiment, polynucleotides coding for a homodimer of the invention are generated by ligating a polynucleotide sequence encoding a polypeptide of the invention to a sequence encoding a linker polypeptide and then further to a synthetic polynucleotide encoding the translated product of the polypeptide in the reverse orientation from the original C-terminus to the N-terminus (lacking the leader sequence) (see, e.g., U.S. Pat. No. 5,478,925, which is herein incorporated by reference in its entirety). In another embodiment, recombinant techniques described herein or otherwise known in the art are applied to generate recombinant polypeptides of the invention which contain a transmembrane domain (or hyrophobic or signal peptide) and which can be incorporated by membrane reconstitution techniques into liposomes (See, e.g., U.S. Pat. No. 5,478,925, which is herein incorporated by reference in its entirety).

II. Pharmaceutical or Physiologically Acceptable Compositions of the Invention

The OBG3 and gOBG3 polypeptide fragments of the invention can be administered to non-human animals and/or humans, alone or in pharmaceutical or physiologically acceptable compositions where they are mixed with suitable carriers or excipient(s). The pharmaceutical or physiologically acceptable composition is then provided at a therapeutically effective dose. A therapeutically effective dose refers to that amount of OBG3 or gOBG3 fragment sufficient to result in prevention or amelioration of symptoms or physiological status of obesity-related diseases or disorders as determined by the methods described herein. A therapeutically effective dose can also refer to the amount of OBG3 or gOBG3 fragment necessary for a reduction in weight or a prevention of an increase in weight or prevention of an increase in the rate of weight gain in persons desiring this affect for cosmetic reasons. A therapeutically effective dosage of an OBG3 or gOBG3 fragment of the invention is that dosage that is adequate to promote weight loss or weight gain with continued periodic use or administration. Techniques for formulation and administration of OBG3 polypeptide fragments may be found in “Remington's Pharmaceutical Sciences,” Mack Publishing Co., Easton, Pa., latest edition.

Other diseases or disorders that OBG3 polypeptide fragments of the invention could be used to treat or prevent include, but are not limited to, obesity and obesity-related diseases and disorders such as obesity, impaired glucose tolerance, insulin resistance, atherosclerosis, atheromatous disease, heart disease, hypertension, stroke, Syndrome X, Noninsulin Dependent Diabetes Mellitus (NIDDM, or Type II diabetes) and Insulin Dependent Diabetes Mellitus (IDDM or Type I diabetes). Diabetes-related complications to be treated by the methods of the invention include microangiopathic lesions, ocular lesions, retinopathy, neuropathy, and renal lesions. Heart disease includes, but is not limited to, cardiac insufficiency, coronary insufficiency, and high blood pressure. Other obesity-related disorders to be treated by compounds of the invention include hyperlipidemia and hyperuricemia. Yet other obesity-related diseases or disorders of the invention include cachexia, wasting, AIDS-related weight loss, cancer-related weight loss, anorexia, and bulimia. The OBG3 or gOBG3 polypeptide fragments may also be used to enhance physical performance during work or exercise or enhance a feeling of general well-being. Physical performance activities include walking, running, jumping, lifting and/or climbing.

The OBG3 or gOBG3 polypeptide fragments or antagonists thereof may also be used to treat dyslexia, attention-deficit disorder (ADD), attention-deficit/hyperactivity disorder (ADHD), and psychiatric disorders such as schizophrenia by modulating fatty acid metabolism, more specifically, the production of certain long-chain polyunsaturated fatty acids.

It is expressly considered that the OBG3 or gOBG3 polypeptide fragments of the invention may be provided alone or in combination with other pharmaceutically or physiologically acceptable compounds. Other compounds useful for the treatment of obesity and other diseases and disorders are currently well-known in the art.

In a preferred embodiment, the OBG3 or gOBG3 polypeptide fragments are useful for, and used in, the treatment of insulin resistance and diabetes using methods described herein and known in the art. More particularly, a preferred embodiments relates to process for the therapeutic modification and regulation of glucose metabolism in an animal or human subject, which comprises administering to a subject in need of treatment (alternatively on a timed daily basis) an OBG or OBG3 polypeptide fragment (or polynucleotide encoding said polypeptide) in dosage amount and for a period sufficient to reduce plasma glucose levels in said animal or human subject.

Further preferred embodiments relate to methods for the prophylaxis or treatment of diabetes comprising administering to a subject in need of treatment (alternatively on a timed daily basis) an OBG or OBG3 polypeptide fragment (or polynucleotide encoding said polypeptide) in dosage amount and for a period sufficient to reduce plasma glucose levels in said animal or human subject.

Routes of Administration.

The composition according to the present invention is preferably administered systemically. The administration can be carried out by methods familiar to a person skilled in the art, for example, intracisternally, intravenously, or peripherally. For the intracisternal or intravenous administration, OBG3 can be suspended, for example, in physiologic saline. However, infusion or bolus injections at regular time intervals are particularly preferred.

Methods of introduction include, but are not limited to, intradermal, intramuscular, intraperitoneal, intravenous, subcutaneous, oral, intrapulmonal, and intrnasal administration. In addition, it may be desireable to introduce the pharmaceutical composition of the invention into the central nervous system by any suitable route, including intrathecal, e.g. intraventricular injection. Intraventricular injection may be faciliatated by an intrventricular cathetar, for example attached to a reservoir, such as an implantable port catheter system.

Furthermore, it may be desirable to administer the pharmaceutical compositions, which are used according to the invention, locally to the area in need of treatment. This may be achieved by, for example, and not by way of limitation, injection by means of a catheter or by means of an inplant, said inplant being of a porous, non-porous or gelatinous material, including membranes, such as sialastic membranes or fibers.

### EXAMPLES

The following Examples are provided for illustrative purposes and not as a means of limitation. One of ordinary skill in the art would be able to design equivalent assays and methods based on the disclosure herein all of which form part of the instant invention.

It should be noted that the term full-length OBG3 polypeptide used throughout the specification is intended to encompass the protein homologs ACRP30 [Scherer, et al., “A novel serum protein similar to C1q, produced exclusively in adipocytes”; J Biol Chem 270, 26746-26749 (1995)], AdipoQ [Hu, et al., “AdipoQ is a novel adipose-specific gene dysregulated in obesity”, J Biol Chem 271, 10697-10703 (1996)] and the human homolog APM1 [Maeda, et al., “cDNA cloning and expression of a novel adipose specific collagen-like factor, APM1 (AdiPose Most abundant Gene transcript 1)”, Biochem Biophys Res Commun 221, 286-289 (1996)] or GBP28 [Nakano, et al., “Isolation and characterization of GBP28, a novel gelatin-binding protein purified from human plasma”, J Biochem (Tokyo) 120, 803-812 (1996)]. OBG3 is also intended to encompass other homologs.

### Example 1

### Demyelination in “Gottingen Mini Pigs”

Lesion:

An experimental model of demyelination has been established in the female adult “Gottingen Mini Pig” (age: 10-14 months, weight 25-30 kg) by stereotaxic injection of lysolecithen (LL) at multiple sites into periventricular subcortical white matter of the brain (usually 2-3 injection sites located in one hemisphere; 5 ul of 1% LL in 0.9% saline per lesion infused over a period of 15 min.). This infusion causes a rapid reduction of myeline sheaths within the diffusion area of the detergent (Blakemore, W. F., Neuropathol. Appl. Neurobiol. 4 (1978) 47-59).

OBG3 Application:

Immediately after LL-application, a stainless steel needle is implanted in the left ventricle space and affixed with an ionomeric bonding bone cement and an osmotic pump (2oo ul volume, infusion rate is connected with the needle and implanted subcutaneously, or a catheter system) is implanted either into the lateral ventricle of the brain or in the subarachinoidal space of the lumer spinal cord.

Ways of application:

1. Chornic infusion of OBG3 via Alzet pump directly intracerebral, beyond the blood brain barrier:
application interval: 7-20 days
concentration of OBG3: 0.4 mg/ml, dissolved in PBS, pH 7.2-7.4
dosages: 0.5 ug/h corresponding to 1.2-4 ug/kg/interval

2. Pulsative injections of OBG3:
After fixation of the catheter with bone cement, the intrathecal catheter is connected with a subcutaneous drug delivery system that is implanted subcutaneously in the upper back region. OBG3 (dissolved in phosphate buffer saline) injected every 2^{nd} day through the skininto the port, starting 2 days after initial LL lesions, by single bolus injections into the port at a dose of 0.05-5 ug/kg body weight (3-350 ug/injection). 3-5 injections were carried out into the cerebrospinal fluid within a period of 6-12 days afer LL-induced demyelination.
Controls:
Controls are carried out by replacement of OBG3 with an equal dose of Cytochrome C applied in the same ways and time intervals/

Evaluation of OBG3 effects:

At the end of the OBG3 (control, respectively) application period, the experimental animals are sacrificed, the brain removed and prepared for histological examination: the areas of interest were studied with routine histological (H.E. staining, Luxol Fast Blue staining), immunocytochemical (myeline basic protein-MBP) and MRI-analysis.

Additional References
1. Shapiro, L. & Scherer, P. E., “The crystal structure of a complement-1q family protein suggests an evolutionary link to tumor necrosis factor”, Curr Biol 8, 335-338 (1998).
2. Kishore, U. & Reid, K. B., “Modular organization of proteins containing C1q-like globular domain”, Immunopharmacology 42, 15-21 (1999).
3. Kavety, B. & Morgan, J. I., “Characterization of transcript processing of the gene encoding precerebellin-1”, Brain Res Mol Brain Res 63, 98-104 (1998).
4. Satoh, F. et al., “Cerebellin and cerebellin mRNA in the human brain, adrenal glands and the tumour tissues of adrenal tumour, ganglioneuroblastoma and neuroblastoma”, J Endocrinol 154, 27-34 (1997).
5. Mazzocchi, G. et al., “Cerebellin enhances in vitro secretory activity of human adrenal gland”, J Clin Endocrinol Metab 84, 632-635 (1999).
6. Kondo, N. & Kondo, J., “Identification of novel blood proteins specific for mammalian hibernation”, J Biol Chem 267, 473-478 (1992).
7. Takamatsu, N., Ohba, K., Kondo, J., Kondo, N. & Shiba, T., “Hibernation-associated gene regulation of plasma proteins with a collagen-like domain in mammalian hibernators”, Mol Cell Biol 13, 1516-1521 (1993).
8. Spiegelman, B. M. & Hotamisligil, G. S., “Through thick and thin: wasting, obesity, and TNF alpha”, Cell 73, 625-627 (1993).
9. Saito, K. et al., “Regulation of gelatin-binding protein 28 (GBP28) gene expression by C/EBP”, Biol Pharm Bull 22, 1158-1162 (1999).
10. Takamatsu, N. et al., “Expression of multiple alpha1-antitrypsin-like genes in hibernating species of the squirrel family”, Gene 204, 127-132 (1997).
11. Ross, S. R., Graves, R. A. & Spiegelman, B. M., “Targeted expression of a toxin gene to adipose tissue: transgenic mice resistant to obesity”, Genes Dev 7, 1318-1324 (1993).
12. Moitra, J. et al., “Life without white fat: a transgenic mouse”, Genes Dev 12, 3168-3181 (1998).
13. Peters, S. J., Dyck, D. J., Bonen, A. & Spriet, L. L., “Effects of epinephrine on lipid metabolism in resting skeletal muscle”, Am J Physiol 275, E300-309 (1998).
14. Rigotti, A., Acton, S. L. & Krieger, M., “The class B scavenger receptors SR-BI and CD36 are receptors for anionic phospholipids”, J Biol Chem 270, 16221-16224 (1995).
15. Hirsch, D., Stahl, A. & Lodish, H. F., “A family of fatty acid transporters conserved from mycobacterium to man”, Proc Natl Acad Sci USA 95, 8625-8629 (1998).
16. Hotamisligil, G. S. et al., “IRS-1-mediated inhibition of insulin receptor tyrosine kinase activity in TNF-alpha- and obesity-induced insulin resistance”, Science 271, 665-668 (1996).
17. Peraldi, P. & Spiegelman, B., “TNF-alpha and insulin resistance: summary and future prospects”, Mol Cell Biochem 182, 169-175 (1998).
18. Kirchgessner, T. G., Uysal, K. T., Wiesbrock, S. M., Marino, M. W. & Hotamisligil, G. S., “Tumor necrosis factor-alpha contributes to obesity-related hyperleptinemia by regulating leptin release from adipocytes”, J Clin Invest 100, 2777-2782 (1997).
19. Uysal, K. T., Wiesbrock S. M., Marino, M. W. & Hotamisligil, G. S., “Protection from obesity-induced insulin resistance in mice lacking TNF- alpha function”, Nature 389, 610-614 (1997).
20. Jensen, D. R. et al.. “Prevention of diet-induced obesity in transgenic mice overexpressing skeletal muscle lipoprotein lipase”, Am J Physiol 273, R683-689 (1997).
21. Levak-Frank, S. et al., “Muscle-specific overexpression of lipoprotein lipase causes a severe myopathy characterized by proliferation of mitochondria and peroxisomes in transgenic mice”, J Clin Invest 96, 976-986 (1995).
22. Scheja, L. et al., “Altered insulin secretion associated with reduced lipolytic efficiency in aP2−/−mice”, Diabetes 48, 1987-1994 (1999).
23. Griffin, M. E. et al., “Free fatty acid-induced insulin resistance is associated with activation of protein kinase C theta and alterations in the insulin signaling cascade”, Diabetes 48, 1270-1274 (1999).
24. Dresner, A. et al., “Effects of free fatty acids on glucose transport and IRS-1-associated phosphatidylinositol 3-kinase activity”, J Clin Invest 103, 253-259 (1999).
25. Kelley, D. E., Goodpaster, B., Wing, R. R. & Simoneau, J. A., “Skeletal muscle fatty acid metabolism in association with insulin resistance, obesity, and weight loss”, Am J Physiol 277, E1130-1141 (1999).
26. Roden, M. et al., “Mechanism of free fatty acid-induced insulin resistance in humans”, J Clin Invest 97, 2859-2865 (1996).
27. Deacon, C. F. et al., “Dipeptidyl peptidase IV inhibition reduces the degradation and clearance of GIP and potentiates its insulinotropic and antihypcrglycemic effects in anesthetized pigs”, Diabetes 50, 1588-1597 (2001).

1-11. (canceled)

12. A method of increasing the rate of regeneration of myelin sheaths of axons comprising the step of contacting an oligodendrocyte progenitor cell with a composition comprising a polypeptide sequence comprising the amino acid sequence of SEQ ID NO:6 or a biologically active fragment thereof.

13. The method according to claim 12, wherein said method is used to treat multiple sclerosis.

14. The method according to claim 12, wherein said method is used to treat hereditary leukodystrophies selected from the group consisting of metachromatic leukodystrophy, Refsum's disease, adrenoleukodystrophy, Krabbe's disease, phenylketonuria, Canavan disease, Pelizaeus-Merzbacher disease, and Alexander's disease.

15. The method according to claim 12, wherein said polypeptide comprises an amino acid sequence selected from the group consisting of amino acids 84-244, 85-244, 101 to 244, 102-244, or 103-244 of SEQ ID NO:6.

16. The method according to claim 15, wherein said polypeptide is administered to an individual in need thereof.

17. The method according to claim 12, wherein said polypeptide comprises an amino acid sequence selected from the group consisting of amino acids 166-193, amino acids 166-176, or amino acids 167-176 of SEQ ID NO:6 or a biologically active fragment thereof.

18. The method according to claim 17, wherein said polypeptide is administered to an individual in need thereof.

19. The method according to claim 12, wherein said polypeptide is administered to an individual in need thereof.

20. A method of increasing the rate of cellular division of oligodendrocyte progenitor cells in which said oligodendrocyte progenitor cells are contacted with a composition comprising a polypeptide sequence comprising the amino acid sequence of SEQ ID NO:6 or a biologically active fragment thereof.

21. The method according to claim 20, wherein said method is used to treat multiple sclerosis.

22. The method according to claim 20, wherein said method is used to treat hereditary leukodystrophies selected from the group consisting of metachromatic leukodystrophy, Refsum's disease, adrenoleukodystrophy, Krabbe's disease, phenylketonuria, Canavan disease, Pelizaeus-Merzbacher disease, and Alexander's disease.

23. The method according to claim 20, wherein said polypeptide comprises an amino acid sequence selected from the group consisting of amino acids 84-244, 85-244, 101 to 244, 102-244, or 103-244 of SEQ ID NO:6.

24. The method according to claim 23, wherein said polypeptide is administered to an individual in need thereof.

25. The method according to claim 20, wherein said polypeptide comprises an amino acid sequence selected from the group consisting of amino acids 166-193, amino acids 166-176, or amino acids 167-176 of SEQ ID NO:6 or a biologically active fragment thereof.

26. The method according to claim 25, wherein said polypeptide is administered to an individual in need thereof.

27. The method according to claim 20, wherein said polypeptide is administered to an individual in need thereof.

28. A method of increasing the rate of migration of oligodendrocyte progenitor cells from the germinal centers to demyelinated axons in the CNS in which said oligodendrocyte progenitor cells are contacted with a composition comprising a polypeptide sequence comprising the amino acid sequence of SEQ ID NO:6 or a biologically active fragment thereof.

29. The method of according to claim 28, wherein said method is used to treat multiple sclerosis.

30. The method according to claim 28, wherein said method is used to treat hereditary leukodystrophies selected from the group consisting of metachromatic leukodystrophy, Refsum's disease, adrenoleukodystrophy, Krabbe's disease, phenylketonuria, Canavan disease, Pelizaeus-Merzbacher disease, and Alexander's disease.

31. The method according to claim 28, wherein said polypeptide comprises an amino acid sequence selected from the group consisting of amino acids 84-244, 85-244, 101 to 244, 102-244, or 103-244 of SEQ ID NO:6.

32. The method according to claim 31, wherein said polypeptide is administered to an individual in need thereof.

33. The method according to claim 31, wherein said polypeptide comprises an amino acid sequence selected from the group consisting of amino acids 166-193, amino acids 166-176, or amino acids 167-176 of SEQ ID NO:6 or a biologically active fragment thereof.

34. The method according to claim 33, wherein said polypeptide is administered to an individual in need thereof.

35. The method according to claim 31, wherein said polypeptide is administered to an individual in need thereof.

36. A method of inducing oligodendrocyte progenitor cells to differentiate into mature dendrocytic cells at sites of demyelination in axons in the CNS in which said oligodendrocyte progenitor cells are contacted with a composition comprising a polypeptide sequence comprising the amino acid sequence of SEQ ID NO:6 or a biologically active fragment thereof.

37. The method according to claim 36, wherein said method is used to treat multiple sclerosis.

38. The method according to claim 36, wherein said method is used to treat hereditary leukodystrophies selected from the group consisting of metachromatic leukodystrophy, Refsum's disease, adrenoleukodystrophy, Krabbe's disease, phenylketonuria, Canavan disease, Pelizaeus-Merzbacher disease, and Alexander's disease.

39. The method according to claim 36, wherein said polypeptide comprises an amino acid sequence selected from the group consisting of amino acids 84-244, 85-244, 101 to 244, 102-244, or 103-244 of SEQ ID NO:6.

40. The method according to claim 39, wherein said polypeptide is administered to an individual in need thereof.

41. The method according to claim 36, wherein said polypeptide comprises an amino acid sequence selected from the group consisting of amino acids 166-193, amino acids 166-176, or amino acids 167-176 of SEQ ID NO:6 or a biologically active fragment thereof.

42. The method according to claim 41, wherein said polypeptide is administered to an individual in need thereof.

43. The method according to claim 36, wherein said polypeptide is administered to an individual in need thereof.

44. A method of treating diseases in which demyelination of nerve fibers occurs, comprising the administration of a composition comprising an amino acid sequence selected from the group consisting of amino acids 84-244, 85-244, 101 to 244, 102-244, 103-244, 166-193, 166-176, or 167-176 of SEQ ID NO:6 or a biologically active fragment thereof, and a pharmaceutical carrier.

45. A method for the production of a pharmaceutical composition comprising the addition of a pharmaceutical carrier to an amino acid sequence selected from the group consisting of amino acids 84-244, 85-244, 101 to 244, 102-244, 103-244, 166-193, 166-176, or 167-176 of SEQ ID NO:6 or a biologically active fragment thereof is used as an essential component of the composition.


SEQUENCE LISTING (7 sequences)

SEQ ID NO: 1 - DNA, 1152 bp, mus musculus
gaattcggcacgagggatgctactgttgcaagctctcctgttcctcttaatcctgcccagtcatgccgaagatgacgttactacaactgaagagctagctcctgctttggtccctccacccaagggaacttgtgcaggttggatggcaggcatcccaggacattctggccacaatggcacaccaggccgtgatggcagagatggcactcctggagagaagggagagaaaggagattcaggtcttcttggtcctaagggtgagacaggagatgttggaatgacaggagctgaagggcctcggggcttccccggaacccctggcaggaaaggagagcctggagaagccgcttatgtgtatcgctcaggcttcagtgtggggctggagacccgcgtcactgttcccaatgtacccattcgctttactaagatcttctacaaccaacagaatcattatgacaacagcactggcaagttctactgcaacattccgggactctactacttctcttaccacatcacggtgtacatgaaagatgtgaaggtgagcctcttcaagaaggacaaggccgttctcttcacctacgaccagtatcaggaaaagaatgtggaccaggcctctggctctgtgctcctccatctggaggtgggagaccaagtctggctccaggtgtatggggatggggaccacaatggactctatgcagataacgtcaacgactctacatttactggctttcttctcttccatgataccaactgactgcaactactcatagcccatacaccaggagaatcatggaacgtcgacacactttcagcttagtttgagagattgattttattgcttagtttgagagtcctgagtattatccacacgtgtactcacttgttcattaaacgactttataaaaaataatttgtgttcctagtccagaaaaaaaggcactccctggtctccacgactcttacatggtagcaataacagaatgaaaatcacatttggtatgggggcttcacaatattcgcatgactgtctggaagtagaccatgctatttttctgctcactgtacacaaatattgttcacataaaccctataatgtaaatatgaaatacagtgattatcttctcaaaaaaaactcgtgccgaattc

SEQ ID NO: 2 - protein, 247 aa, mus musculus
MLLLQALLFLLILPSHAEDDVTTTEELAPALVPPPKGTCAGWMAGIPGHSGHNGTPGRDGRDGTPGEKGEKGDSGLLGPKGETGDVGMTGAEGPRGFPGTPGRKGEPGEAAYVYRSGFSVGLETRVTVPNVPIRFTKIFYNQQNHYDNSTGKFYCNIPGLYYFSYHITVYMKDVKVSLFKKDKAVLFTYDQYQEKNVDQASGSVLLHLEVGDQVWLQVYGDGDHNGLYADNVNDSTFTGFLLFHDTN

SEQ ID NO: 3 - DNA, 1276 bp, mus musculus
ctctaaagattgtcagtggatctgacgacaccaaaagggctcaggatgctactgttgcaagctctcctgttcctcttaatcctgcccagtcatgccgaagatgacgttactacaactgaagagctagctcctgctttggtccctccacccaagggaacttgtgcaggttggatggcaggcatcccaggacatcctggccacaatggcacaccaggccgtgatggcagagatggcactcctggagagaagggagagaaaggagatgcaggtcttcttggtcctaagggtgagacaggagatgttggaatgacaggagctgaagggccacggggcttccccggaacccctggcaggaaaggagagcctggagaagccgcttatatgtatcgctcagcgttcagtgtggggctggagacccgcgtcactgttcccaatgtacccattcgctttactaagatcttctacaaccaacagaatcattatgacggcagcactggcaagttctactgcaacattccgggactctactacttctcttaccacatcacggtgtacatgaaagatgtgaaggtgagcctcttcaagaaggacaaggccgttctcttcacctacgaccagtatcaggaaaagaatgtggaccaggcctctggctctgtgctcctccatctggaggtgggagaccaagtctggctccaggtgtatggggatggggaccacaatggactctatgcagataacgtcaacgactctacatttactggctttcttctctaccatgataccaactgactgcaactacccatagcccatacaccaggagaatcatggaacagtcgacacactttcagcttagtttgagagattgattttattgcttagtttgagagtcctgagtattatccacacgtgtactcacttgttcattaaacgactttataaaaaataatttgtgttcctagtccagaaaaaaaggcactccctggtctccacgactcttacatggtagcaataacagaatgaaaatcacatttggtatgggggcttcacaatattcgcatgactgtctggaagtagaccatgctatttttctgctcactgtacacaaatattgttcacataaaccctataatgtaaatatgaaatacagtgattactcttctcacaggctgagtgtatgaatgtctaaagacccataagtattaaagtggtagggataaattggaaaaaaaaaaaaaaaaaaagaaaaactttagagcacactggcggccgttactag

SEQ ID NO: 4 - protein, 247 aa, mus musculus
MLLLQALLFLLILPSHAEDDVTTTEELAPALVPPPKGTCAGWMAGIPGHPGHNGTPGRDGRDGTPGEKGEKGDAGLLGPKGETGDVGMTGAEGPRGFPGTPGRKGEPGEAAYMYRSAFSVGLETRVTVPNVPIRFTKIFYNQQNHYDGSTGKFYCNIPGLYYFSYHITVYMKDVKVSLFKKDKAVLFTYDQYQEKNVDQASGSVLLHLEVGDQVWLQVYGDGDHNGLYADNVNDSTFTGFLLYHDTN

SEQ ID NO: 5 - DNA, 4517 bp, Homo sapiens
ctgattccataccagaggggctcaggatgctgttgctgggagctgttctactgctattagctctgcccgggcatgaccaggaaaccacgactcaagggcccggagtcctgcttcccctgcccaagggggcctgcacaggttggatggcgggcatcccagggcatccgggccataatggggccccaggccgtgatggcagagatggcacccctggtgagaagggtgagaaaggagatccaggtcttattggtcctaagggagacatcggtgaaaccggagtacccggggctgaaggtccccgaggctttccgggaatccaaggcaggaaaggagaacctggagaaggtgcctatgtataccgctcagcattcagtgtgggattggagacttacgttactatccccaacatgcccattcgctttaccaagatcttctacaatcagcaaaaccactatgatggctccactggtaaattccactgcaacattcctgggctgtactactttgcctaccacatcacagtctatatgaaggatgtgaaggtcagcctcttcaagaaggacaaggctatgctcttcacctatgatcagtaccaggaaaataatgtggaccaggcctccggctctgtgctcctgcatctggaggtgggcgaccaagtctggctccaggtgtatggggaaggagagcgtaatggactctatgctgataatgacaatgactccaccttcacaggctttcttctctaccatgacaccaactgatcaccactaactcagagcctcctccaggccaaacagccccaaagtcaattaaaggctttcagtacggttaggaagttgattattatttagttggaggcctttagatattattcattcatttactcattcatttattcattcattcatcaagtaactttaaaaaaatcatatgctatgttcccagtcctggggagcttcacaaacatgaccagataactgactagaaagaagtagttgacagtgctattttgtgcccactgtctctcctgatgctcatatcaatcctataaggcacagggaacaagcattctcctgtttttacagattgtatcctgaggctgagagagttaagtgaatgtctaaggtcacacagtattaagtgacagtgctagaaatcaaacccagagctgtggactttgttcactagactgtgcccttttatagaggtacatgttctctttggagtgttggtaggtgtctgtttcccacctcacctgagagccattgaatttgccttcctcatgaattaaaacctcccccaagcagagcttcctcagagaaagtggttctatgatgaagtcctgtcttggaaggactactactcaatggcccctgcactactctacttcctcttacctatgtcccttctcatgcctttccctccaacggggaaagccaactccatctctaagtgctgaactcatccctgttcctcaaggccacctggccaggagcttctctgatgtgatatccacttttttttttttttgagatggagtctcactctgtcacccaggctggagtacagtgacacgacctcggctcactgcagcctccttctcctgggtccaagcaattattgtgcctcagcctcccgagtagctgagacttcaggtgcattccaccacacatggctaatttttgtatttttagtagaaatggggtttcgtcatgttggccaggctggtctcgaactcctggcctaggtgatccacccgcctcgacctcccaaagtgctgggattacaggcatgagccaccatgcccagtcgatatctcactttttattttgccatggatgagagtcctgggtgtgaggaacacctcccaccaggctagaggcaactgcccaggaaggactgtgcttccgtcacctctaaatcccttgcagatccttgataaatgcctcatgaagaccaatctcttgaatcccatatctacccagaattaactccattccagtctctgcatgtaatcagttttatccacagaaacattttcattttaggaaatccctggtttaagtatcaatccttgttcagctggacaatatgaatcttttccactgaagttagggatgactgtgattttcagaacacgtccagaatttttcatcaagaaggtagcttgagcctgaaatgcaaaacccatggaggaattctgaagccattgtctccttgagtaccaacagggtcagggaagactgggcctcctgaatttattattgttctttaagaattacaggttgaggtagttgatggtggtaaacattctctcaggagacaataactccagtgatgtttttcaaagattttagcaaaaacagagtaaatagcattctctatcaatatataaatttaaaaaactatctttttgcttacagttttaaattctgaacaatttctcttatatgtgtattgctaatcattaaggtattattttttccacatataaagctttgtctttttgttgttgttgttgtttttaagatggagtttccctctgttgccaggctagagtgcagtggcatgatctcggcttactgcaacctttgcctcccaggtttaagcgattcttctgcctcagcctcccgagtagctgggaccacaggtgcctaccaccatgccaggctaatttttgtatttttagtaaagacagggtttcaccatattggccaggctggtctcgaactcctgaccttgtgatctgcccgcctccattgtgttgttatttgtgagaaagatagatatgaggtttagagagggatgaagaggtgagagtaagccttgtgttagtcagaactctgtgttgtgaatgtcattcacaacagaaaacccaaaatattatgcaaactactgtaagcaagaaaaataaaggaaaaatggaaacatttattcctttgcataatagaaattaccagagttgttctgtctttagataaggtttgaaccaaagctcaaaacaatcaagacccttttctgtatgtccttctgttctgccttccgcagtgtaggctttaccctcaggtgctacacagtatagttctagggtttccctcccgatatcaaaaagactgtggcctgcccagctctcgtatccccaagccacaccatctggctaaatggacatcatgttttctggtgatgcccaaagaggagagaggaagctctctttcccagatgccccagcaagtgtaaccttgcatctcattgctctggctgagttgtgtgcctgtttctgaccaatcactgagtcaggaggatgaaatattcatattgacttaattgcagcttaagttaggggtatgtagaggtattttccctaaagcaaaattgggacactgttatcagaaataggagagtggatgatagatgcaaaataatacctgtccacaacaaactcttaatgctgtgtttgagctttcatgagtttcccagagagacatagctggaaaattcctattgattttctctaaaatttcaacaagtagctaaagtctggctatgctcacagtctcacatctggtgggggtgggctccttacagaacacgctttcacagttaccctaaactctctggggcagggttattcctttgtggaaccagaggcacagagacagtcaactgaggcccaacagaggcctgagagaaactgaggtcaagatttcaggattaatggtcctgtgatgctttgaagtacaattgtggatttgtccaattctctttagttctgtcagcttttgcttcatatattttagcgctctattattagatatatacatgtttagtattatgtcttattggtgcatttactctcttatcattatgtaatgtccttctttatctgtgataattttctgtgttctgaagtctactttgtctaaaaataacatacgcactcaacttccttttctttcttccttcctttctttcttccttcctttctttctctctctctctttccttccttccttcctccttttctctctctctctctctctctctcttttcttgacagactctcgttctgtggccctggctggagttcagtggtgtgatcttggctcactgctacctctaccatgagcaattctcctgcctcagcctcccaagtagctggaactacaggctcatgccactgcgcccagctaatttttgtatttttcgtagagacggggtttcaccacattcgtcaggttggtttcaaactcctgactttgtgatccacccgcctcggcctcccaaagtgctgggattacaggcatgagccatcacacctggtcaactttcttttgattagtgtttttgtggtatatctttttccatcatgttactttaaatatatctatattattgtatttaaaatgtgtttcttacagactgcatgtagttgggtataatttttatccagtctaaaaatatctgtcttttaattggtgtttagacaatttatatttaataaaatggtggaatttaaa

SEQ ID NO: 6 - protein, 244 aa, Homo sapiens
MLLLGAVLLLLALPGHDQETTTQGPGVLLPLPKGACTGWMAGIPGHPGHNGAPGRDGRDGTPGEKGEKGDPGLIGPKGDIGETGVPGAEGPRGFPGIQGRKGEPGEGAYVYRSAFSVGLETYVTIPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 7 - DNA, 20966 bp, Homo sapiens
  misc_feature 1..4811: 5′ regulatory region
gctgatctgctgcctcagccttcccaaagtgctgtaatttattaggcataagccactgtgcctgcctagtgttgtacattctgtgggttttgacaattgtatgcatctacatgtatgtaccatttatagtattcctgtttttaattttagccattctagtaggcatgtagtgatatctcatggtgattttaatttgcgtttccgtaatggttaataatgctgaacatctttgcatgtgcttgtttgtcatttgtgtttcctacttggtgaaataattgttcatgtcctttgtccattttctaattgaatttttttttaccatttagttttgagatttctttatacaatctagatccaaatctcttgtctcaaatatggtttgcaaatacattcctctaattcatatattgccttttcctcctcttaacaggatgtttcacagagcaaaagttttagttttgttgaaatctcacttttcatttttttctttagtggattgtgcttttgttgtcatatgtaagaactcttcactggccctagatccttgtattggtttcctaagattgccatagcaaatcaccatgaacttagtgacaaaaagacagaaatttattttcacttcctactgtgggcagactagacgttaattattttcatgtatgctcattcctatgacatctttctgatataataattatagttattcttaagcttcacccttttttctattagctttgttaccttgggtgtcactttttcttttttgacattgtgacctatgccagatcatgtctgttagtacttagccctccattcacctctccataatcccttttgtattcctggagcttgatgcctgaaatgacacatcctacattcctttgccagatgggtaccagttagcttgtgcacatgggagacaaccgtgaaaagactgaagtggggaagaagggaggagctgttgtgtttcagtgagcgcccttggcagtggcggtgacagtggctcctgttcagtggcaatggtggagcagctagcaagacatgcagtaagcgcaggctcataggctatggtccaggagcagtcaccgattcctggtctttaggcaatatcatctccctttgcttctccagcctttctaaaattattgtaccttgactagtacaattttttagtattgggggtagtccaaggacacaggctttaaaaagtatgaattcagggttgcctacctgcattgactgcgcttgaatcatgatggccttctggtcggtggcaggaggtgacagtccaaatcatgcagtagcaaaccagatacttaaattatcatctgagatacttcagaagtacagccgtagccataccttcagaagagataaagaaatgttctcctggccaggcgcggtggctcacgcctgtcattccagcactttgggaggccgagggggtggatcacctgaggtcgggagttcgagaccagcctgaccaacatggggaaaccctgtctctactaaaaatacaaaattagcggggcgtggtggcacatgcccataatcccagctactcgggaggctaaggcaggataatcgcttgaacctgagaggcagaggttgcggtgaactgagatcatgccatagtactccagcctgggcaacaagagtgaaactccatctcaaaaaaaaaaaaaagaaaaaaagataaagaaatgttctcctttcttgccatttctaggggtttggggatggcgtacattgctgcagggcgtgctcactctaccatcttgctccaatctttatttttcaaaatacagtgcttatgcttggttacttcagttaagattatttttaaaaatcataattaagcaaaaatatatggccatgcttaaacatatttaagataaattaagtgatttggcctgtttcagtatcccaactcacatgctaacaggggcttgacctgtagctacggtaccctggaggaaatgatcgcatttatttggttatttcggtctaagtagtaatagttctgtcctgggaaaaagactagcctcaaggcatttctgattgaatgtttttcaattacagtctttaaaccagtatgccacagaactggctctttccacatgacggcctttgtggtgggtggcagattgccctgaggcctcgcaaaatgctaggctttcacaatgtcactgactgacagccaggcccagcacagtcttggtgtgattgtggggctaaagttattccaccttgtgcaatagctacagccttctctaaccagctgcattcttataaagttagaagaaaatacttttttttttttgagatggattctcgctctgttgcccaggctggagtgcaatggtgcgatctcggctcgctgcaacctccgcctcctgggttcaaacgattctcctccctcagacccccgagtagctgggattgcaggtgcctgccaccacgcccggctaacttttttgtatttttagtggagacggggtttcaccatcttcgtcaggctggtctcagactcctgacctcaagtgatctgcccgcctcagcctcccaaaatgctgggattacaggcatgagctactgtgcccggccaaagaaaatactttttatgccagccctgaaactaccctgaagcacatacatcaaccttgaggcctcacactccatcaagaggggtgaagggcatgaggaattagaaagcatagggatttttagttagacagatctggttcaaatcctagacttgtgccttgaacaaattatttaccctcattgaactctagattcattatttgtaaaatgaaagacaataatagttatctccaaaggaaagttgaatatgatcattcatttattcattaattcaacatttattattgcctactttgtgccaggttctattctaggaactaagggatacaactttgaataggcaaaatctctgctctcctgaagtttactttttttttttttttttgagacagagtttcactcttgtcacccaggctggagcgcaatggtgctcttggctcactgcaacctccacctcctgggttcaagtgattctcttgtctcagcctcccaagtagctgggactacaggtatgtgccaccacgcccggctatttctgcatttttagtagagatggggtttcaccatgttggccagactggtctcaaactcctgatctcaggtgatatgcctgtcttggccttccaaagtactgggattacaggcctgagccactgcacctgacctgaagtttatgttctattaaatagcaacagacagtaacataaaccaaaaataaataggaaaacaccataacaaaaatcaaacagtgatataattgagagttgcttctatttctttttgttgtcttcttggttcaatcagcctgctaaactatatggaacctcattttcatgggccacttatttaagccgggggaccttggaaagtctctcatgtctctcatctcaacggcctaatgtgacttctcttgaaatatttggacattagcaggaagctgaggctttacatcagatctttactttaatggtggacttgactttactggtagatttttaggctctgtgtggactgtggagatgatatctggggggcaggcagacacttgccctgcctctgtctgagaaaattctgttttggatgtcttgttgaagttggtgctggcatcctaagcccttgctggggtcgtaatttaattcatcagaatgtgtggcttgcaagaaccggctcagatcctgcscttcaaaaacaaaacatgagcgtgccaagaaagtccaaggtgttgaatgttgccacttcaagcctaaactttctaggaacacctaagtgggtggcagcttccagttctccaggctgcttctaggccagagctgggttccacaagagacagaataggcatatatatgcttaaggaactggaaaaacaggctctctctctctcacaaacacacacacacacataccaaggtagctgtcaaaatgttatccgaaattttggaaccaaaaaatcttgaaagatggtattccaatatcacattttatgtaagttttctattatattagattcaaattacgattcgaggccacaagctttaagaattcagggcctttttaacttgccaagccccacaccactccaggaacttccccacaccccagttctcagaattcatgtgcaaggtctttcctaaatccagggtccaggtcagagagtggaggatgtgctctatttcttacctgattgcagacccctctgacagtgctcccttctgaagcactcactgtctgaacgtacacagtctcagacttaatcatgcacagtgagcaagactgtggtgtgataattggcgtccctgacttattagggcaaatctatgggagggggagacctcctggaccactgagcaattaattcatttacattaggaagtttctccgtcagatgcaggaaaaaaatcttgttttcctgctgtggttttgacttttgccccatcttctgttgctgttgtaggaggcaaaataagggtcaaggcctggaaacacaagtgctttgactgaagctccacttggcttccgaagcccaagctgggttgtaccaggttccctagggtgcaggctgtgggcaactgccagggacatgtgcctgcccaccggcctctggccctcactgagttggccaatgggaaatgacaattgtgaggtggggactgcctgcccccgtgagtaccaggctgttgaggctgggccatctcctcctcacttccattctgactgcagtctgtggttctgattccataccagagggtaagagcaattctgtgaagttccaggctgggtgggggatgcatgcatagcctctggctgggatcacccaggctctcccgtccgtagtagtgtgggagtggatacaggtggatactctggtcagagcagcactggtggaggcagatatgcactgggcttcttcctccgttctcccacagccccaagagagaaagggttatttcagacattccttctaagatgcatggaaccattctgaattttacccagttcgctctgtagcaggatacctattgagaaaaagttagggtcagtaaggtggaagggtctgtccacagatgaagtccaattcgattaagggggataagggaatacattgtctcttagcttgaccaggtagggcaaaggaagaagcatatatgaaggcagcttcagaaaagtcaagctgagcactgacttcagactggaattaggaatccagctctgccactttattctactcagcaaatatttactgagcaaattctatgggctagacagtggattgggttcacaagatacaatgagtgtgacatggttgttgtctatggatttggggatatatgtaggtatagggatatcttacaaggtaatcaagaggttctaatgaggccagccatggtggctcacacctgtaatcccagcaatttgggagaccgaggcgggtggatcacctgaggtcaggagttccagactagcctgaccaacatggtgaaaccccgcctctaccaaaaatacaaaaattagttgggcgtgatggcaggtgcctgtaatcccagcttctcgggaggctgaggcaggagaattgtctgaacctgggaggcagaggttgcagtgagccgagattgttgccactgcattccagcctgggtgacagagcgagactttgtgtcaaaaaaaaaaaaaaaaagaaagaaaagaaaaagaggctctaatgagataaaatgagaaaagcctggcatgtagtggcaacttatgaaaaattgtaattaaaaaaaaacattttctgacagaagaaactggatctacctggtttttctgaagcctaatcctgctcgccccagtgagtgctgtttctgaggcatcctggttgttttgagctgtggatgctgaaggttagagtgggagggattttagaggttaggtctgcccctcttgtgttagaggacatggatccctggtctggagaggttctggtttttggatcaagcctcacaaggggtggcaccaactcactcctaggaactccgctagaaggaaggccagctctgcctaattcggttggggagatgggggtccctttatgctagcagaatatgtccgaaggagcatgatggtgtcagctttgttcatgaaggccagtggtacacagggagcccggcagcttcctcagcagtccctgctgccactcttccttaagtcttgaggagtctttttttggcacaatctcagctcactgcaacctccgcctcccaggttcaagcgattctcctgcctcagtctcccaagtagctgagactacaggcatgcgccaccacgcccagctaatttttatatttttagtagagatggggttcaccatattggccaggatggtctcgatctcttgacctcatattccacctgcctcggcctcccaaagtgctggtattacaggtgtgagccactgcgcctggccgaggagtcttaagctgagatcacagcattgcactccagcctgggcaaaaagagcaaaactccatctcaaaaaaaaaaaaaaaatagacacaagactggctccttgtcttttttggggacagggtctcactctatcacccaggctggagtgcagtggtgcaatcacagctcactgcagcctcgatttcccaggctcaagtgaccctcccatcttagcctcctgagtagctgggactacaggtgtgtgcaaccatgcctggctaatttttaaaaattttttgtagagatgaggtctcactatattggctggggggcctcaaactcctgggctcagcagtcctcccacctcagcctcccaaaaggctgggattatatgcttgctctttttaaggtggctgtagggacaaactttccacctactccttgtcaagccagtggaccggtggtcccagacatacggctaaagtcaagaggtgatgtcttttggagagatactttcaatcaggaatttcaatcagaaattcaatcatgtggagagagacttatcctaaaaatgtggtggtgcgtgggatgctctgttttattagttccttgacagtatgtatgtgtgtgagtgtgtgtgtgtgcgcgcgcacactcatttggatgggtgtgtatgtgtgtgggggggtggtgcgtacgtatgtggatgtgtggatgtggtgtgtgggtgtgcgcgtgcataggtggaggtgtgtgtatgggtgcgggtatgtgtgtgtgttgggcatggagatattgacagctctcccagggctgagtgaaggctttcgggcaaagctcctgggagctaggcaaagctgagttgattcctggttatgccatttattattgggttgcaccgtgtgaaactgccaatattctacactttgacttttatttatttttatttttattttttttgagacagagtttcacacttgtcacctaggctggagtgcagtggcgcgatctcagctcactgcaacctctgcctcatggattcaagtgattctcctgcctcagcctcccaagtagctggaattacaggtgcccgctaccacgcctgactaatttttgtatttttggtagagacgggatttcaccatgttgtccaggctggtctgaaactcctgacatcaggtaatccacccacctcagcctcccaaagtgctgggattacaggcatgagccactgcgcccggcccattttgacttttaaaaatgggagtttgatataattcaatccagtggttgaattagctagcatcgttccctctccaagtctcaggttctcctacacgttagagtcaaaagcagggctatgggaagattaagtaaaataaattttgaaaatgccttatgaaaattacactccaaagaactcgcgccagtgtcagtgttctcatgttcctcatctcacatgatcacatttcgcggattaggaagctgagtctgagaagctccgtgtagtgctttttcggaggcaccgtgatgtgatggaaggctcactcgttaggaagtcagaacagagtctctgagggatcatttccttaatctgtcagtttcctcatctctgaagttgggctcatttccttccttcatggagttattgtaaagatgaagataaataacgtgtaaaatctagcatgggaactggcttctataaggttctaataagtgcattcctactccttcccctcagccttcccatttgtaaaagcaaggcaggggtgaggtgatttctggggctccttttggctctgacatttgaggattttgtatccttttttttttcagagtcttgctctgtcacccaggttggagtgcagctcaatgcaaattccgcctcccaggctcaagcaattcttatgtctcagcctcctgagtacctgggattacaggcaggcaccaccacccccagctaattttttgtattttcagtagagacggggttttgccatattggccaggctggtcttgaactcctgacttcatgtgacccacccatctcagcctcccaaagtgctgagatgacaggtgtgagctaccgtgcctggccaattttgtgtgctttaatgcccttttctgctggaagagttggcaccaggtttggtgatctctttcccccacacggctctgcctcctgccagtcccagaggggaccctgtccttgcatttcacaggattctgctgttgcaactgaaattccagtaggtcaaagtgaaatttctcatacactttaacatgaagataaatgatcacagtatggccctttaggatcctgagaacatcacggtcatcccctggtataattttaaaagcagatgaatccatgcctgtgcgaggtttgccaggaaagccagtgctgggattacagtggaagtctttttatgctacttttttcttgtatccctcaccccatggggtggcatattgaaaggcaggatgtgtgaccacgatacttttctcctcctggactatgtctaagagtctgttattgggttctgaagatcagagtttaatttccgactcctctctgtgtagctctgggatcttggaaagccacttaacctttctgaagtcccctttcctcatctctaaaatgcatacactcatcactaacatttactgagcactgacatgtgccagacaccattctaagcattttacacagactacaccatttgatcttccaacaaacagaacactgaaacgcattacaggtcagaacaaatgatttgtgcctaagcaccaagaccgtagagcccgtgctccctattctaccctatcctgtctctcaaaatgattgtgagaatcgaatgagacactaggtgagaaaagggttttataaatagcattttaaaaattttttaaagtccacaaaatttttaattttaatacagataaaatagatccctttgttttataaaaagtaacaaaatttgttatacaacaactatgttatttattaattttgcctttttgtatgctgccaggaaagaaacattaagaaatcttaaattgattatggtgaatcagaaggtctgcctggactttttattgctctaactgtacagctgatcatactacctcatttttttttatgacacttcaagggtgcgcttagcttcatcactccttcgttgccaaaagctttgtgaccaaaaacaattaagcagattcctgagtcactaaatgacacataaccagagttgagacttaggaacttttagtgccatgctaagcccacagggacacaacaaatagcattttacaaaggcaaagaattgtgacacttgagatttagcttgttgatccttgtaaaagttttctttttaggcataattgagttttagatcatagtactcactattacttagtaataatttttttctgatagaaatacagtgtaacaggccgggcgcagtggctcatgcctgtaatcccagcactttgggaggccgaggcgggcggatcacttgaggtcaggagtttgagaccagcccggccaacatggtgaaatcccatctctactaaaaatacaaaaaattagccaggtgtggtcgtggattcctgtgatcccagctacttgggagggtgaggcaggagcatcagttgaacccaggaggcggaggttgcagtgagccaagatggtgccattgcactccagcctgggccacaaagcgagactccacttcagaaacaaaaaaaaaaagagagagagagaaaagaaggaaggaaggaaggaaggaaggaaagaaggaaggaaggaaggaaagaaggaaggaaggaaggaaggaaagaaggaaggaaggaaagaaggaaggaaggaaagaaggaaggaaggaaggaaggaagggtaacaagcaaagtgtaacaatggcaatatctaaaaaaataggtatttttatatgtttgtcgttttatatatatgacccccactttagagatgaggaaactgagagattaaggaaacgatccctgagagactctgttctgacttccaaatcggtgagctttccatcgcatcacggtgcctccgaaagcatgacacggagcttctcagacttagcttcctaatccgctaaacgggattatgtgagatgaggaacatgagaacgctgacatgggtgagggttccttggagtatcattttcatgtggcattttcaaaacttattttacctaatcttcccaaagccctgcttttgactctaatgtgtctcctgagacttggagagcgcaagatgctagcgacagagcaagactccatctccagataaataaataagtaaaataaaaaagaacacaaataattttgaaaatttttttgaaaattaggcacgtttgcactgaccttcaattgttattaattgctggtttcccacccagaattaagttggaatgcaactttcttttacaatcagagtccgttcttggtcttggaaacttctgaggctcctgtgctaatcccactcttgtatttttggcacctctaccccgtgccactgtcatggaacccaggctgatcgcacctattagtggagaaatmtgtccataatactgaagtttggggacaaacagtgttcccttagggtaggagaaagagatctttatttttaacaaagggggaggagccagaaaactccagagacccctgagtttgccctctctccaaggtttggggtaagccccccgtcaccctttatctctggggctttcacatattctggattctctcctcctgtttcccagcagaaaaggatggagcctcacagattcttcccatttctggagaaaaacatgcatggagctcaaagttcttctcaggagttttattgccaaagccataataagaaagggtggaggtgacaagcagtgaggaagtttaaagatgcatgaaatctgtaaagtctcagaacaagaattctcctaaaatgcaaaaggggctttgctggtctccccttggcttctcatgtagctcacctcttttttcttatcttgagactagtcaaacctaagctgtttctcattttatttccagaagctattgagaacactctcctgaattcttcaaattcagtagagggcgacaaatgtacatataaatgatggtagtgggtcttaaataaagactcatgacacctaaaggggcagcacctgagtctgattgcacctgtttctgttgctgtttctgtctctcttctctctgtctgccatttcattatcaatggttactttacttataagatcatattagaacctgatatttgataaatgatgcatcagatctatagtgagagaaaaaattaatgcaattaaaggtgttgtaacagctagtcttcaagtggggagaaatcatttgagtaccttaggtcacagcttacatcaaaacaaaaaatcagagctacattaaaaagtgaaattttaactatatcaaacaatagaaaaaaacagaagaaaattgaatacttactaaatcttagcatgaataagaactgtttaacacttagaggcaaggactgggcgtggtggctcatgcttttaatcccaggactttgggagcccaaggcgggcggatcacctgaggtcaggagtttgagactagcctggccaacatggtgaaaccccgtctctactaaaaaatgcaaaaattagctgcgtgtggtggtgcatgcctgtaatctcagctacttgggaggctaaggcatgagaatcgcttgaacctgggaggtggaggctgtagtgagccgagattgtgccactgcactacagcctgggtgacagtgtgaaatcctctctctcaaaaaaaaaaaaaaaaaaaagcaaactagagcagtgaggtaccattatttcctttgctcactaaactgacaacacacaaatgttttttataatacccaaagctgatgagggtagttaaggtatgcccttttatacacacactaatgatgtactactggttggcagtataacatatgctgccatgtggggatatgtatcaggagacttaaaaatgtgcataccttttggtccagtaatttacttctgggaatctgtcataacagaataataatcttggggaaagctacatgcctaaggatatttaaaatattatttaaaaatcaaagtataatttcttacagaatataaaataatattttaaaatgaaaatatgctaaaagtttgatgaaatataaatggtcaaatatatattgattatatccacttactagactagcactcactctgagacgttaaaaatagtcattataaaaactagaaaatgccaaagacaaaataaaggaataaagttttacataaagtatgattccactatgtttaaaaataaacagagacattcttggagttgagtattgttttcttttctgtcatgtccaaagaactatataactattatttttaatgaactatatatgtaatatatacatatagtttatatgtatatacaaaatttatctcatatatatgataaagatgaaagatgagttggatgtgccacgtgaagtgggtagtatagaaacccaggtaatggggcataggagtgggattccagataccaggcccatgtttttggggtgagattgccaatcacggtctttcttccatccctcacagaggagtaggtttgtcttcaacaaaccttcagttgtcctgaagacaaacctaattctggagacttcatataatctagaagagacaagcaaactgatgaaaaatagtgaatttttaaggtaaaataaagtacatggactacactttgtttagaatcagattcttgggattaaccacattaacccacagagggtcttagtgatgcctctaatccaggatcctaggacctatttctctctgtgagatgctttctcccaactccttggtgagagtgggaagactaagacctcagcaatctgaggtggaggcctaagatccccctaagatcggaggcagaatctgagaggggataaaagtccctatacctgtattgggcccttttctgggagggggatatcaaagaatgattttgagacagggaggcttttgactacctgtgccacttgagctctttgctagggctccagaatacatatttcaaatacattccccctccctccttccttccctcttccactcttcctttttatcttcctttcttcttttccttcctccttcccttcctttctctggctctctcatgatttcttttcctcattataaaagtgcttatttagtccctactctgctattagtgtgttagtctttgtcccctggtacttgctgtttaatggagaaatgggtgagcaaaacagaaattacagcagagtgcaataatagagctaagccaggtgtataaatccattctcacactgctgtaaaaaactactgggtaatttataaagaaaagaggtttaattgactcacagttccacaggctgtacaggaagcatggctggggaggcctcagaaaacttacaatcatggtggaagaaagagcgaaggggaagcaagcacatcacacagcagcaggagagagagagagaaagagagagagagagaatataggggaagtgctacacactttcaaccagatcttgtgagaattcacctactatcatgagaacagcaagggataagtctgcctccatgattcagtcacctcctaccaggccccttctccaacacatgtcgacgtgctatttgggtggggacacagacccaaaccatattaccagggcactggagaaacacagaggggaaagaaccagccaaggagtgagatggagaacaaggaggacttcttgaaacagatgacatccaaactgggtcctgaaagctgaatagagattagacaggggaggaggggcagctaaagatggctcaggcaaacaaagggccaggggatatgttcatgggatgatgtgtctctcgttgtctgcttaacacaaggtgagtctctccctccctctctctctctttttctctgtgtgtgtttgtgtgtgtgcatgtgtgcaaatgtaatatacccaatagtcaaacatgtgccccaggagaggggtagaggaagaaagagaatgagagagtaagaaggaggaatagacacagaaaatgagagagaaggggggaaagaaaaagaagaaaggagccagaggagagaagctggttagcattgaatggagcaatctgtgtcatcgtacttgggaaacccaaggatggattcttggcaagtcgactcttggagctttccctgtgcttggtcctgtgctcagacatgggaaaattagaggagtgtcatctgtgcaatcactgaattcataatcttggtgaggaaaggagactacacacagggaataatgctaagtattacagatttcagggcagaaagagatcaaggtgggctgcaatattcagaaaagtcttcctggaaaagttgaatacttagaaagcagctcctagaagtagactctgctgagatggacggagtcctttgtaggtcccaactgggtgtgtgtgtggggtctgtctctccatggcygacagtgcacatgtggattccagggctcaggatgctgttgctgggagctgttctactgctattagctctgcccggkcatgaccaggaaaccacgactcaagggcccggagtcctgcttcccctgcccaagggggcctgcacaggttggatggcgggcatcccagggcatccgggccataatggggccccaggccgtgatggcagagatggcacccctggtgagaagggtgagaaaggagatccaggtaagaatgtttctggcctctttcatcacagacctcctacactgatataaactatatgaagkcattcattattaactaaggcctagacacagggagaaagcaaagcttttttatgttaaccataagcaacctgargtgatttggggttggtcttccaaggatgagtgtagatggtgcctctataaccaagactttggctttgctgcatctgcagctccttttccatcccctttcccatcttcaccctcatccctattcccagtacattcatattctgattcctctttctgtctgcttaacttccatttcacccagtggcattcaaccacatttactgcacaccccctgaaaggctcagtcctgcctttggggaactcttgatctaggtaagatgtctaatgtgcaaggctctgttggtggttaccacaagaaagtctactctaaaaatgtcaaactgaatgtgaacaagtattcaaagtatggagcatagagaaaatrtactcaccgtggacctgatgaagaatgaaggcttcaaggaggaggcagagcttcagctaggccttgaatgatgggtaggcagaatagaggaggagagacatcctagatggagggggtagaattgcaaaaccagggttgatggtgccagcacataaagggctggcagggtggagggtctatgatagagacctataggagataaagatagagttgaaattatgggagcctccatgtctgtgggagatatagaaggaggaggtaacacctctctccttttgggagctcttattggtttcttgatctataagtcaagaaggttgtgagtgggagccacagggatggtaatttaggctgtaaccaacctaggcaggagttctgttctttgtagtcactgaggtcttctcattccttaggtcttattggtcctaagggagacatcggtgaaaccggagtacccggggctgaaggtccccgaggctttccgggaatccaaggcaggaaaggagaacctggagaaggtgcctatgtataccgctcagcattcagtgtgggattggagacttacgttactatccccaacatgcccattcgctttaccaagatcttctacaatcagcaaaaccactatgatggctccactggtaaattccactgcaacattcctgggctgtactactttgcctaccacatcacagtctatatgaaggatgtgaaggtcagcctcttcaagaaggacaaggctatgctcttcacctatgatcagtaccaggaaaataatgtggaccaggcctccggctctgtgctcctgcatctggaggtgggcgaccaagtctggctccaggtgtatggggaaggagagcgtaatggactctatgctgataatgacaatgactccaccttcacaggctttcttctctaccatgacaccaactgatcaccactaactcagagcctcctccaggccaaacagccccaaagtcaattaaaggctttcagtacggttaggaagttgattattatttagttggaggcctttagatattattcattcatttactcattcatttattcattcattcatcaagtaactttaaaaaaatcatatgctatgttcccagtcctggggagcttcacaaacatgaccagataactgactagaaagaagtagttgacagtgctattttgtgcccactgtctctcctgatgctcatatcaatcctataaggcacagggaacaagcattctcctgtttttacagattgtatcctgaggctgagagagttaagtgaatgtctaaggtcacacaagtattaagtgacagtgctagaaatcaaacccagagctgtggactttgttcactagactgtgcccttttatagaggtacatgttctctttggagtgttggtaggtgtctgtttcccacctcacctgagagccattgaatttgccttcctcatgaattaaaacctcccccaagcagagcttcctcagagaaagtggttctatgatgaagtcctgtcttggaaggactactactcaatggcccctgcactactctacttcctcttacctatgtcccttctcatgcctttccctccaacggggaaagccaactccatctctaagtgctgaactcatccctgttcctcaaggccacctggccaggagcttctctgatgtgatatccacttttttttttttttgagatggagtctcactctgtcacccaggctggagtacagtgacacgacctcggctcactgcagcctccttctcctgggtccaagcaattattgtgcctcagcctcccgagtagctgagacttcaggtgcattccaccacacatggctaatttttgtatttttagtagaaatggggtttcgtcatgttggccaggctggtctcgaactcctggcctaggtgatccacccgcctcgacctcccaaagtgctgggattacaggcatgagccaccatgcccagtcgatatctcactttttattttgccatggatgagagtcctgggtgtgaggaacacctcccaccaggctagaggcaactgcccaggaaggactgtgcttccgtcacctctaaatcccttgcagatccttgataaatgcctcatgaagaccaatctcttgaatcccgtatctacccagaattaactccattccagtctctgcatgtaatcagttttatccacagaaacattttcattttaggaaatccctggttttaagtatcaatccttgttcagctggacaatatgaatcttttccactgaagttagggatgactgtgattttcagaacacgtccagaatttttcatcaagaaggtagcttgagcctgaaatgcaaaacccatggaggaattctgaagccattgtctccttgagtaccaacagggtcagggaagactgggcctcctgaatttattattgttctttaagaattacaggttgaggtagttgatggtggtaaacattctctcaggagacaataactccagtgatgttcttcaaagattttagcaaaaacagagtaaatagcattctctatcaatatataaatttaaaaaactatctttttgcttacagttttaaatcctgaacaattctctcttacatgtgtattgctaatcattaaggtattattttttccacatataaagctttgtctttttgttgttgttgttgtttttaagatggagtttccctctgttgccaggctagagtgcagtggcatgatctcggcttactgcaacctttgcctcccaggttcaagcgattcttctgcctcagcctcccgagtagctgggaccacaggtgcctaccaccatgccaggctaatttttgtatttttagtaaagacagggtttcaccatattggccaggctggtctcgaactcctgaccttgtgatctgcccacctccatttttgttgttattttttgagaaagatagatatgaggtttagagagggatgaagaggtgagagtaagccttgtgttagtcagaactctgtgttgtgaatgtcattcacaacagaaaacccaaaatattatgcaaactactgtaagcaagaaaaataaaggaaaaatggaaacatttattcctttgcataatagaaattaccagagttgttctgtctttagataaggtttgaaccaaagctcaaaacaatcaagacccttttctgtatgtccttctgttctgccttccgcagtgtaggctttaccctcaggtgctacacagtatagttctagggtttccctcccgatatcaaaaagactgtggcctgcccagctctcgtatccccaagccacaccatctggctaaatggacatcatgttttctggtgatgcccaaagaggagagaggaagctctctttcccagatgccccagcaagtgtaaccttgcatctcattgctctggctgagttgtgtgcctgtttctgaccaatcactgagtcaggaggatgaaatattcatattgacttaattgcagcttaagttaggggtatgtagaggtattttccctaaagcaaaattgggacactgttatcagaaataggagagtggatgatagatgcaaaataatacctgtccacaacaaactcttaatgctgtgtttgagctttcatgagtttcccagagagacatagctggaaaattcctattgattttctctaaaatttcaacaagtagctaaagtctggctatgctcacagtctcacatctggttggggtgggctccttacagaacacgctttcacagttaccctaaactctctggggcagggttattcctttgtggaaccagaggcacagagagagtcaactgaggccaaaagaggcctgagagaaactgaggtcaagatttcaggattaatggtcctgtgatgctttgaagtacaattgtggatttgtccaattctctttagttctgtcagcttttgcttcatatattttagcgctctattattagatatatacatgtttagtattatgtcttattggtgcatttactctcttatcattatgtaatgtccttctttatctgtgataattttctgtgttctgaagtctactttgtctaaaaataacatacgcactcaacttccttttctttcttccttcctttctttcttccttcctttctttctctctctctctctttccttccttccttcctccttttctttctctctctctctctctctctttttttgacagactctcgttctgtggccctggctggagttcagtggtgtgatcttggctcactgctacctctaccatgagcaattctcctgcctcagcctcccaagtagctggaactacaggctcatgccactgcgcccagctaatttttgtatttttcgtagagacggggtttcaccacattcgtcaggttggtttcaaactcctgactttgtgatccacccgcctcggcctcccaaagtgctgggattacaggcatgagccatcacacctggtcaactttcttttgattagtgtttttgtggtatatctttttccatcatgttactttaaatatatctatattattgtatttaaaatgtgtttcttacagactgcatgtagttgggtataatttttatccagtctaaaaatatctgtcttttaattggtgtttagacaatttatatttaataaaattgttgaatttaagatggatgactgttttatttgtttgctgttcaccacttctgttttattctctttccagaattcttttggattgtttaaatatttcataatattttatcttaatttatttattgggtatttgcctatatctctttgtggtattttttagtggttgcttgagggattacaatgtacttaacttttcacagtgtgcataaagttaatattttgccacttgcagtaaaccgtagaaggcttataatcatattagtacctctatccactttcttttatgttgtagttgtcatatatattacatctatatacactgaaacattataggcaatgttatgatttttgcattcgtcagtcatatatatattttaaagaatttaagaggagaaaaatacatattcagatattcatcat